(19) United States
(12) Patent Application Publication
(10) Pub. No.: US 2007/0077596 A1
(43) Pub. Date: Apr. 5, 2007

(54) TRACE INCORPORATION OF FLUORESCENT MONOMER FACILITATING QUALITY CONTROL OF POLYMERIZATION REACTIONS

(75) Inventors: Arkadi Krainev, Fremont, CA (US); Wenxi Huang, Fremont, CA (US); Charanjit Saini, Milpitas, CA (US)

Correspondence Address: TOWNSEND AND TOWNSEND AND CREW LLP, TWO EMBARCADERO CENTER, 8TH FLOOR, SAN FRANCISCO, CA, 94111-3834, US

(73) Assignee: Ciphergen Biosystems, Inc.

(21) Appl. No.: 11/465,942

(22) Filed: Aug. 21, 2006

Related U.S. Application Data
(63) Continuation of application No. 10/850,698, filed on May 21, 2004.
(60) Provisional application No. 60/472,879, filed on May 22, 2003.

(51) Int. Cl. G01N 33/53; C12M 1/34; G01N 33/50

(52) U.S. Cl. 435/7.1; 436/86; 435/287.2

(57) ABSTRACT

The present invention provides a polymer that includes a trace amount of a luminescent moiety. The polymers have unique properties that make them ideally suited for use in diverse analyses, including desorption/ionization mass spectrometry of analytes. The invention also provides a device that incorporates the polymeric compositions of the inventions, methods of using the device to detect, quantify and identify analytes, and a method of preparing a device of the invention. The luminescent moiety provides a detectable tracer that allows for a rapid, simple quality control assay of devices that incorporate the polymer.

### CROSS-REFERENCES TO RELATED APPLICATIONS

This application is a continuation of U.S. application Ser. No. 10/850,698, filed May 21, 2004, which claims the benefit of U.S. Provisional Patent Application No. 60/472,879, filed May 22, 2003, the teachings of each of the aforementioned applications are herein incorporated by reference.

### BACKGROUND OF THE INVENTION

Bioassays are used to probe for the presence and/or the quantity of an analyte material in a biological sample. In surface based assays, the analyte is quantified by its capture and detection on a solid support. One example of a surface-based assay is a DNA microarray. The use of DNA microarrays has become widely adopted in the study of gene expression and genotyping due to the ability to monitor large numbers of genes simultaneously (Schena et al., Science 270:467-470 (1995); Pollack et al., Nat. Genet. 23:41-46 (1999)). More than 100,000 different probe sequences can be bound to distinct spatial locations across the microarray surface, each spot corresponding to a single gene (Schena et al., Tibtech 16:301-306 (1998)). When a fluorescent-labeled DNAn analyte sample is placed over the surface of the array, individual DNA strands hybridize to complementary strands within each array spot. The level of fluorescence detected quantifies the number of copies bound to the array surface and therefore the relative presence of each gene, while the location of each spot determines the gene identity. Using arrays, it is theoretically possible to simultaneously monitor the expression of all genes in the human genome. This is an extremely powerful technique, with applications spanning all areas of genetics. (For some examples, see, the Chipping Forecast supplement to Nature Genetics 21 (1999)). Arrays can also be fabricated using other binding moieties such as antibodies, proteins, haptens or aptamers, in order to facilitate a wide variety of bioassays in array format.

Other surface-based assays include microtitre plate-based ELISAs in which the bottom of each well is coated with a different antibody. A protein sample is then added to each well along with a fluorescent-labeled secondary antibody for each protein. Analyte proteins are captured on the surface of each well and secondarily labeled with a fluorophore. The fluorescence intensity at the bottom of each well is used to quantify the amount of each analyte molecule in the sample. Similarly, antibodies or DNA can be bound to a microsphere such as a polymer bead and assayed as described above. Once again, each of these assay formats is amenable for use with a plurality of binding moieties as described for arrays.

Other bioassays are of use in the fields of proteomics, and the like. For example, cell function, both normal and pathologic, depends, in part, on the genes expressed by the cell (i.e., gene function). Gene expression has both qualitative and quantitative aspects. That is, cells may differ both in terms of the particular genes expressed and in terms of the relative level of expression of the same gene. Differential gene expression is manifested, for example, by differences in the expression of proteins encoded by the gene, or in post-translational modifications of expressed proteins. For example, proteins can be decorated with carbohydrates or phosphate groups, or they can be processed through peptide cleavage. Thus, at the biochemical level, a cell represents a complex mixture of organic biomolecules.

One goal of functional genomics (“proteomics”) is the identification and characterization of organic biomolecules that are differentially expressed between cell types. By comparing expression, one can identify molecules that may be responsible for a particular pathologic activity of a cell. For example, identifying a protein that is expressed in cancer cells but not in normal cells is useful for diagnosis and, ultimately, for drug discovery and treatment of the pathology. Upon completion of the Human Genome Project, all the human genes will have been cloned, sequenced and organized in databases. In this “post-genome” world, the ability to identify differentially expressed proteins will lead, in turn, to the identification of the genes that encode them. Thus, the power of genetics can be brought to bear on problems of cell function.

Differential chemical analyses of gene expression and function require tools that can resolve the complex mixture of molecules in a cell, quantify them and identify them, even when present in trace amounts. The current tools of analytical chemistry for this purpose are presently limited in each of these areas. One popular biomolecular separation method is gel electrophoresis. Frequently, a first separation of proteins by isoelectric focusing in a gel is coupled with a second separation by sodium dodecyl sulfate-polyacrylamide gel electrophoresis (SDS-PAGE). The result is a map that resolves proteins according to the dimensions of isoelectric point (net charge) and size (i.e., mass). Although useful, this method is limited in several ways. First, the method provides information only about two characteristics of a biomolecule-mass and isoelectric point (“pI”). Second, the resolution power in each of the dimensions is limited by the resolving power of the gel. For example, molecules whose mass differ by less than about 5% or less than about 0.5 pI are often difficult to resolve. Third, gels have limited loading capacity, and thus limited sensitivity; one often cannot detect biomolecules that are expressed in small quantities. Fourth, small proteins and peptides with a molecular mass below about 10-20 kDa are not observed.

The use of mass spectrometric methods is replacing gels as the method of choice for bioassays. Efforts to improve the sensitivity of assays have resulted in the application of a number of mass spectrometric formats to the analysis of samples of biological relevance. In addition to the innovations in mass spectrometric techniques, substrates that adsorb an analyte (“chips”) have also developed and the early designs have been improved upon.

Particularly useful methods of performing bioassays rely on the use of an adsorbent chip in conjunction with mass spectrometry. Prior investigators, have reported a variety of techniques for analyte detection using mass spectroscopy, but these techniques suffered because of inherent limitations in sensitivity and selectivity of the techniques, specifically including limitations in detection of analytes in low volume, undifferentiated samples (Hillenkamp, Bordeaux Mass Spectrometry Conference Report, pp. 354-62 (1988); Karas and Hillenkamp, Bordeaux Mass Spectrometry Conference Report, pp. 416-17 (1988); Karas and Hillenkamp, Analytical Chemistry, 60:2299 2301(1988); Karas, et al., Biomed. Environ. Mass Spectrum 18:841-843 (1989)). The use of laser beams in time-of-flight mass spectrometers is shown, for example, in U.S. Pat. Nos. 4,694,167; 4,686,366, 4,295,046, and 5,045,694, incorporated herein by reference.

Exemplary mass spectrometric formats include matrix assisted laser desorption/ionization mass spectrometry (MALDI), see, e.g., U.S. Pat. No. 5,118,937 (Hillenkamp et al.) and U.S. Pat. No. 5,045,694 (Beavis and Chait), and surface enhanced laser desorption/ionization mass spectrometry (SELDI), see, e.g., U.S. Pat. No. 5,719,060 (Hutchens and Yip), incorporated herein by reference.

There exists a need for reliable, low cost biochips that allow for the rapid detection and characterization of micro-quantities of cellular tissue, peptides, genetic material, small organic molecules, etc. for use in research as well as in the diagnosis of disease(s) or the existence of predetermined conditions. Recently, analytical devices have experienced a miniaturization trend similar to that experienced in the electronics industry with the advent of integrated circuits. Many of the same principles that have led to smaller and smaller micro processor devices have shrunk the size of a chemistry lab to a device no larger than the size of a microscope slide. The techniques are all aimed at producing a device having different, discreet areas that interact in a predictable manner with an analyte or mixture of analytes. These areas, or devices, are formed using a number of techniques, including photo patterning methods, such as photolithography, which is a direct descendant from techniques used in the manufacture of micro processor chips; micro machining, where tiny channels are machined into a chip to hold various test media; and other methods of precisely depositing test media upon chips in a precisely defined pattern.

While these methods allow for the manufacture of acceptable biochips, they do have certain drawbacks. One significant drawback is the sophistication and expense of photo patterning, micro machining and micro-media deposition devices that are capable of producing biosensor chips including multiple individual regions that interact with analyte. Additionally, the use of these methods requires extreme precision in the deposition of the materials that interact with analyte. The variability in the methods used to deposit the material that interacts with the analyte can also lead to significant quality control issues, since a single biochip consists of a plurality of separate regions, each of which requires testing or verification.

Accordingly, there is a need for an improved biochip or device and method of manufacturing the same, which provides inexpensive biosensor devices that are manufactured using cost-effective methods and devices. Additionally, there is a need for a biochip that is highly reliable due to improved quality assurance procedures performed during the manufacture of the biochip. Finally, a biochip is needed that can utilize the same manufacturing methods for a wide variety of biochip formats. The present invention meets these and other needs in the art.

### BRIEF SUMMARY OF THE INVENTION

It has now been discovered that a polymer that includes subunits derived from a monomeric luminescent moiety that is useful in desorption/ionization mass spectrometric analyses. The polymer of the invention is unique in that it provides an analyte adsorption gel that is versatile in structure and in the method of its manufacture. Moreover, devices that incorporate the luminescent polymer can be submitted to a simple, rapid and uniform quality control process to insure the integrity of the polymer incorporated into the device. Thus, the consistency of the polymer characteristics of sensors, probes, chips and other devices manufactured using the luminescent polymer is readily confirmed for a plurality of devices from a single production batch. Additionally, or alternatively, the consistency of the devices between separate production batches is readily assessed.

Moreover, the present invention provides compositions and devices that enhance the accuracy and the reproducibility of polymer distribution on an analytical device, such as a chip. Additionally, analyses using the luminescent polymer of the invention are more consistent from chip to chip, because the analyses make use of chips on which the consistency of the distribution of the polymer on the chip is confirmed.

The polymeric material comprises a luminescent monomer, incorporated within the polymeric framework, that emits luminescence at a selected wavelength. The luminescent properties of the polymer allow its distribution on a device to be ascertained by visualizing the distribution of the luminescence on the device. For example, when the device includes a plurality of “spots” of the polymer on a surface, the luminescent polymer allows one to determine if each region of the surface that requires a “spot” has polymer at that region. Moreover, where it is desired that the spots have a particular shape, utilizing the luminescent polymer of the invention allows one to determine whether the “spot” has the desired shape.

The luminescent polymer can be essentially a homopolymer (optionally cross-linked) that includes only a trace amount of the luminescent monomer. Alternatively, the luminescent polymer can be formed through the polymerization of monomers of different structure in addition to the luminescent material. Moreover, the luminescent polymer can be formed from monomers that include binding functionalities that bind to one or more analytes.

The luminescent polymer of the invention is easily prepared by art-recognized polymerization methods. For example, a solution of a luminescent monomer and a solution of a non-luminescent monomer are deposited onto a substrate, e.g, a chip, and subsequently polymerized. Alternatively, the polymer is preformed and subsequently deposited onto the substrate. The amount and identity of the luminescent monomer can be varied to produce a polymer having a desired property or set of properties. Moreover, luminescent species of more than one structure can be utilized. Thus, according to the present invention it is possible to “tune” the properties of the polymer by varying the nature and concentration of the constituents of the polymeric matrix.

In addition to the chemical properties, the morphology of the polymer can be varied as well. For example, the polymer can be a film or it can be formed under suspension or emulsion polymerization conditions to form beads or particles of the polymer. Moreover, the polymer can be made non-porous, microporous, or macroporous materials by means of porogens.

The polymer is useful to prepare chips for desorption/ionization mass spectrometric analysis. Thus, in a first aspect, the invention provides a device that includes a substrate having a surface; and a polymeric material attached to the surface. The polymeric material includes a luminescent species covalently bonded within the polymeric framework.

Also provided is a method of preparing a chip of the invention. The method includes depositing onto a chip a polymer that includes a covalently bound luminescent moiety having the analyte-receiving properties set forth above. The polymer can be formed prior to its deposition onto the chip or in situ on the chip.

In yet another aspect, the present invention provided a method of analyzing a sample. The method includes desorbing and ionizing the sample from a chip that includes a luminescent moiety covalently bound within the polymeric framework.

Moreover, as discussed above, the invention provides a method for the quality control of a device that includes a polymer bound to a substrate, such as a biochip. The method includes interrogating the device using a method that detects luminescence to determine the distribution of luminescence originating from the luminescent polymer bound to the substrate. The method is of use for determining the lateral distribution of the polymer across a region of the substrate as well as the thickness of the polymer on the substrate. The method is compatible with high-throughput techniques.

The quality control method is also of use to detect failure of polymerization, i.e., the luminescent monomer is not incorporated into the polymeric framework in an expected amount. The expected amount of luminescence incorporation can be an amount that is calculated from the known amount of luminescent monomer included in the polymerization mixture. Alternatively, the expected amount can be any amount that is greater than the detection threshold of the device utilized to detect the luminescence.

In another embodiment, the invention provides a method of confirming that an analyte is bound to the polymer of the invention. The method includes contacting the polymer with an analyte to which a quencher moiety is bound. When bound to the polymer, the quencher and the luminescent moiety will interact, quenching the light emitted from the luminescent moiety, thereby confirming the intimate interaction of the polymer and the analyte.

Other objects, advantages and aspects of the present invention will be apparent from the detailed description that follows.

### BRIEF DESCRIPTION OF THE DRAWINGS

FIG. 1 is a collection of structures of representative anion exchange (positively charged) binding moieties of use in preparing the chips of the invention.

FIG. 2 is a collection of structures of representative cation exchange (negatively charged) binding moieties of use in preparing the chips of the invention.

FIG. 3 is an exemplary reaction scheme for preparing a cation exchange polymer of the invention.

FIG. 4 is an exemplary reaction scheme for preparing an anion exchange polymer of the invention.

FIG. 5 is a view of a chip of the invention.

FIG. 6 is a graphical display of the change in fluorescence intensity of a polymer of the invention as the ratio of fluorescent monomer to non-fluorescent monomer increases.

FIG. 7 is a bar graph display of the fluorescence intensity for a series of substrates and polymers deposited onto the substrates. In Spot AE, the fluorescent polymer is doped with 0.0025% of the fluorescent monomer. In Spot BF, the fluorescent polymer is doped with 0.001% fluorescent monomer. In Spot CG, the fluorescent polymer is doped with 0.0005% fluorescent monomer. In Spot DH, the fluorescent polymer includes no fluorescent monomer.

FIG. 8 is a single-stage ion optic, linear, constant-energy TOF mass spectrometer. Two ions M₁ and M₂, where M₂ is heavier than M₁, are accelerated to constant energy. Ion TOF is dependent upon m/z, acceleration potential V, acceleration distance s, and free flight distance x.

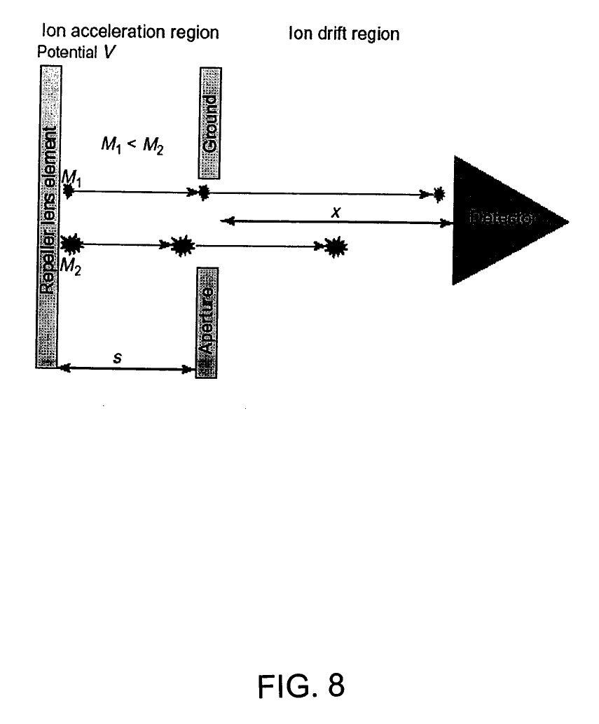

FIG. 9 is a parallel extraction, LDI, linear TOF mass spectrometer. Both constant extraction and PIE modes are illustrated; PS=power supply.

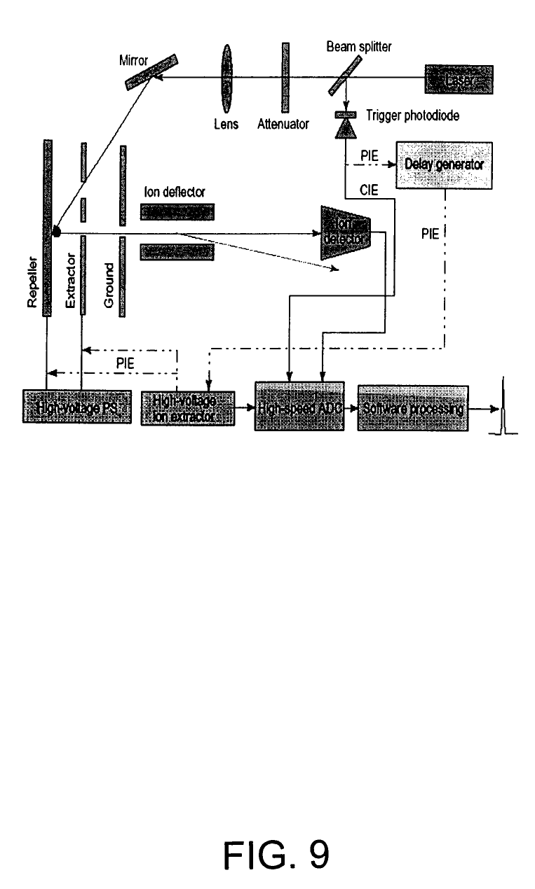

FIG. 10 is an orthogonal extraction TOF device with reflectron analyzer.

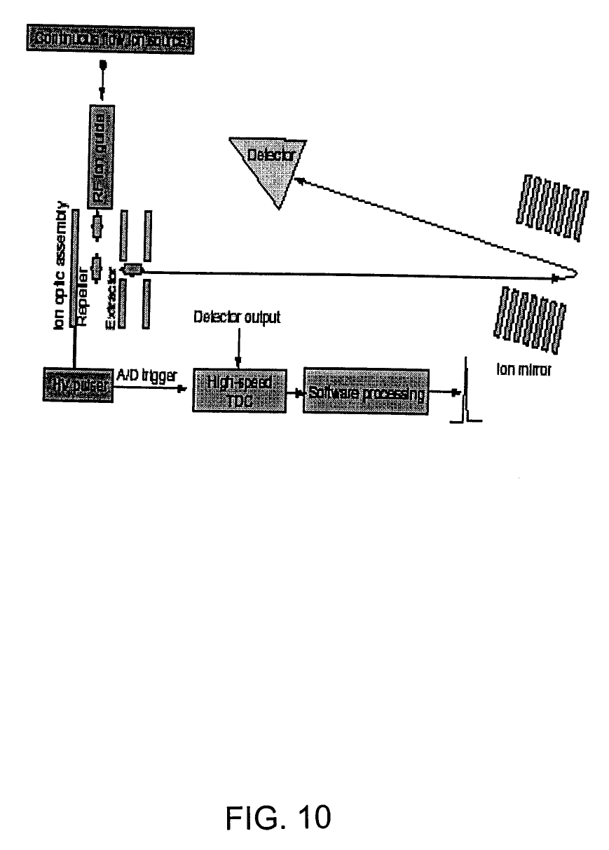

FIG. 11 is an orthogonal electrospray TOF device equipped with quadrupole ion guide providing collisional cooling (q0). Two other quadrupole regions are included for MS/MS purposes (Q1 and q2). TLF performed in the ion optic assembly corrects for initial spatial distribution, whereas the ion mirror corrects for velocity disparities.

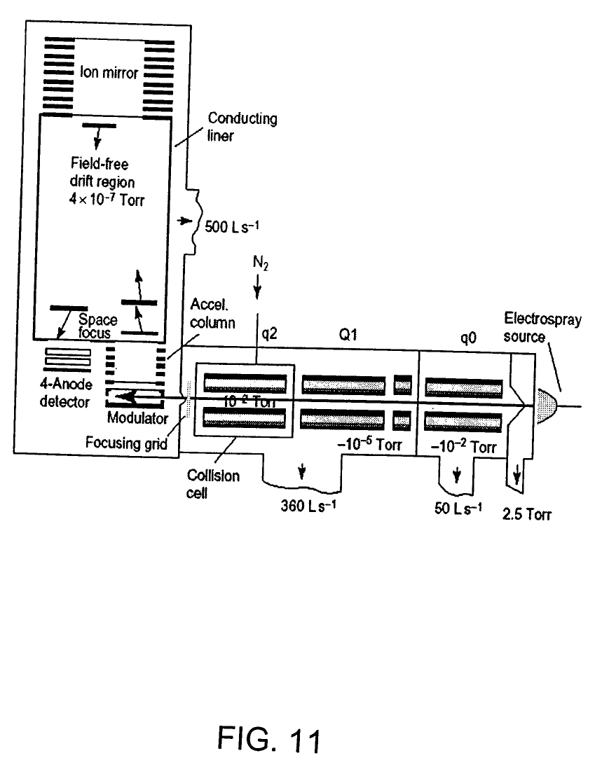

### DETAILED DESCRIPTION OF THE INVENTION

Abbreviations and Definitions

As used herein, “MALDI” refers to Matrix-Assisted Laser Desorption/Ionization.

“SELDI,” as used herein refers to Surface Enhanced for Laser Desorption/Ionization.

“SEND,” as used herein refers to Surface Enhanced for Neat Desorption.

As used herein, “TOF” stands for Time-of-Flight.

As used herein, “MS” refers to Mass Spectrometry.

As used herein “MALDI-TOF MS” refers to Matrix-assisted laser desorption/ionization time-of-flight mass spectrometry.

“Q10” refers to a biochip that is formed by the polymerization of [3-(methacryloylamino)propyl]trimethylammonium chloride onto a cytonix-coated 100 μm grit blasted aluminum substrate that is prepared by CVD (chemical vacuum deposition) of methacryloxypropyl trimethoxysilane onto the substrate.

As used herein, the term “operative contact,” refers to a relationship between an analyte labeled with a quencher moiety and a luminescent polymer.

As used herein, the term “analyte desorption/ionization” refers to converting an analyte into the gas phase as an ion.

The term “matrix” refers to a plurality of generally acidic, energy absorbing chemicals (e.g., nicotinic or sinapinic acid) that assist in the desorption (e.g., by laser irradiation) and ionization of the analyte into the gaseous or vapor phase as intact molecular ions.

As used herein, “energy absorbing molecule, or moiety (EAM)” refers to a light absorbing species that, when presented on the surface of a probe element (as in the case of SEND), facilitate the neat desorption of molecules into the gaseous or vapor phase for subsequent acceleration as intact molecular ions.

As used herein, “desorption” refers to the departure of analyte from the surface and/or the entry of the analyte into a gaseous phase.

As used herein, “ionization” refers to the process of creating or retaining on an analyte an electrical charge equal to plus or minus one or more electron units.

Where substituent groups are specified by their conventional chemical formulae, written from left to right, they equally encompass the chemically identical substituents which would result from writing the structure from right to left, e.g., —CH₂O— is intended to also recite —OCH₂—; —NHS(O)₂— is also intended to represent. —S(O)₂HN—, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above.

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR′C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

Each of the above terms is meant to include both substituted and unsubstituted forms of the indicated radical.

As used herein, the term “heteroatom” is meant to include oxygen (O), nitrogen (N), sulfur (S) and silicon (Si).

“Analyte,” as utilized herein refers to the species of interest in an assay mixture. Exemplary analytes include, but are not limited to cells and portions thereof, enzymes, antibodies and other biomolecules, drugs, pesticides, herbicides, agents of war and other bioactive agents.

The term “substance to be assayed” as used herein means a substance, which is detected qualitatively or quantitatively by the process or the device of the present invention. Examples of such substances include antibodies, antibody fragments, antigens, polypeptides, glycoproteins, polysaccharides, complex glycolipids, nucleic acids, effector molecules, receptor molecules, enzymes, inhibitors and the like.

More illustratively, such substances include, but are not limited to, tumor markers such as α-fetoprotein, carcinoembryonic antigen (CEA), CA 125, CA 19-9 and the like; various proteins, glycoproteins and complex glycolipids such as β₂-microglobulin (β₂ m), ferritin and the like; various hormones such as estradiol (E₂), estriol (E₃), human chorionic gonadotropin (hCG), luteinizing hormone (LH), human placental lactogen (hPL) and the like; various virus-related antigens and virus-related antibody molecules such as HBs antigen, anti-HBs antibody, HBc antigen, anti-HBc antibody, anti-HCV antibody, anti-HIV antibody and the like; various allergens and their corresponding IgE antibody molecules; narcotic drugs and medical drugs and metabolic products thereof, and nucleic acids having virus- and tumor-related polynucleotide sequences.

The term, “assay mixture,” refers to a mixture that includes the analyte and other components. The other components are, for example, diluents, buffers, detergents, and contaminating species, debris and the like that are found mixed with the analyte. Illustrative examples include urine, sera, blood plasma, total blood, saliva, tear fluid, cerebrospinal fluid, secretory fluids from nipples and the like. Also included are solid, gel or sol substances such as mucus, body tissues, cells and the like suspended or dissolved in liquid materials such as buffers, extractants, solvents and the like.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a BHQ, a fluorophore or another moiety.

“Peptide” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid but, which functions in a manner similar to a naturally occurring amino acid.

“Therapeutic agent” or “drug” refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “therapeutic agent” and “drug” encompass both the inactive drug and the active metabolite.

The term “binding functionality” as used herein means a moiety, which has an affinity for a certain substance such as a “substance to be assayed,” that is, a moiety capable of interacting with a specific substance to immobilize it on the chip of the invention. Binding functionalities can be chromatographic or biospecific. Chromatographic binding functionalities bind substances via charge-charge, hydrophilic-hydrophilic, hydrophobic-hydrophobic, van der Waals interactions and combinations thereof. Biospecific binding functionalities generally involve complementary 3-dimensional structures involving one or more of the above interactions. Examples of combinations of biospecific interactions include, but are not limited to, antigens with corresponding antibody molecules, a nucleic acid sequence with its complementary sequence, effector molecules with receptor molecules, enzymes with inhibitors, sugar chain-containing compounds with lectins, an antibody molecule with another antibody molecule specific for the former antibody, receptor molecules with corresponding antibody molecules and the like combinations. Other examples of the specific binding substances include a chemically biotin-modified antibody molecule or polynucleotide with avidin, an avidin-bound antibody molecule with biotin and the like combinations.

The term “detection means” as used herein refers to detecting a signal produced by the immobilization of the substance to be assayed onto the binding functionality by visual judgment or by using an appropriate external measuring instrument depending on the signal properties.

The term “attached,” and “immobilized” are used interchangeably herein and they encompass interactions including, but not limited to, covalent bonding, ionic bonding, electrostatic interactions, hydrogen bonding, hydrophobic-hydrophobic interaction, hydrophilic-hydrophilic interaction, chemisorption, physisorption and combinations thereof.

The term “independently selected” is used herein to indicate that the groups so described can be identical or different.

The term “biomolecule” or “bioorganic molecule” refers to an organic molecule typically made by living organisms. This includes, for example, molecules comprising nucleotides, amino acids, sugars, fatty acids, steroids, nucleic acids, polypeptides, peptides, peptide fragments, carbohydrates, lipids, and combinations of these (e.g., glycoproteins, ribonucleoproteins, lipoproteins, or the like).

The term “biological material” refers to any material derived from an organism, organ, tissue, cell or virus. This includes biological fluids such as saliva, blood, urine, lymphatic fluid, prostatic or seminal fluid, milk, etc., as well as extracts of any of these, e.g., cell extracts, cell culture media, fractionated samples, or the like.

As used herein, “quenching group” refers to any fluorescence-modifying group of the invention that can attenuate at least partly the light emitted by a fluorescent group. This attenuation is referred to herein as “quenching”. Hence, illumination of the fluorescent group in the presence of the quenching group leads to an emission signal that is less intense than expected, or even completely absent. Quenching typically occurs through energy transfer between the fluorescent group and the quenching group.

As used herein, “energy transfer” refers to the process by which the fluorescence emission of a fluorescent group is altered by a fluorescence-modifying group. If the fluorescence-modifying group is a quenching group, then the fluorescence emission from the fluorescent group is attenuated (quenched). Energy transfer can occur through fluorescence resonance energy transfer, or through direct energy transfer. The exact energy transfer mechanisms in these two cases are different. It is to be understood that any reference to energy transfer in the instant application encompasses all of these mechanistically-distinct phenomena.

As used herein, “energy transfer pair” refers to any two molecules that participate in energy transfer. Typically, one of the molecules acts as a fluorescent group, and the other acts as a fluorescence-modifying group. The preferred energy transfer pair of the instant invention comprises a fluorescent group and a quenching group. There is no limitation on the identity of the individual members of the energy transfer pair in this application. All that is required is that the spectroscopic properties of the energy transfer pair as a whole change in some measurable way if the distance between the individual members is altered by some critical amount.

“Energy transfer pair” is used to refer to a group of molecules that form a single complex within which energy transfer occurs. Such complexes may include, for example, two fluorescent groups, which may be different from one another and one quenching group, two quenching groups and one fluorescent group, or multiple fluorescent groups and multiple quenching groups. In cases where there are multiple fluorescent groups and/or multiple quenching groups, the individual groups may be different from one another.

As used herein, “fluorescence-modifying group” refers to a molecule of the invention that can alter in any way the fluorescence emission from a fluorescent group. A fluorescence-modifying group generally accomplishes this through an energy transfer mechanism. Depending on the identity of the fluorescence-modifying group, the fluorescence emission can undergo a number of alterations, including, but not limited to, attenuation, complete quenching, enhancement, a shift in wavelength, a shift in polarity, and a change in fluorescence lifetime. One example of a fluorescence-modifying group is a quenching group.

“Fluorescence resonance energy transfer” or “FRET” is used interchangeably with FET, and refers to an energy transfer phenomenon in which the light emitted by the excited fluorescent group is absorbed at least partially by a fluorescence-modifying group. FRET depends on an overlap between the emission spectrum of the fluorescent group and the absorption spectrum of the quenching group. FRET also depends on the distance between the quenching group and the fluorescent group.

“Moiety” refers to the radical of a molecule that is attached to another moiety.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm² to 50 mJ/mm². Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Adsorption” refers to detectable non-covalent binding of an analyte to an adsorbent or capture reagent.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-methoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Solid support” refers to a solid material which can be derivatized with, or otherwise attached to, a chemical moiety, such as a capture reagent, a reactive moiety or an energy absorbing species. Exemplary solid supports include chips (e.g., probes), microtiter plates and chromatographic resins.

“Chip” refers to a solid support having a generally planar surface to which a chemical moiety may be attached. Chips that are adapted to engage a probe interface are also called “probes.”

“Biochip” refers to a chip to which a chemical moiety is attached. Frequently, the surface of the biochip comprises a plurality of addressable locations, each of which location has the chemical moiety attached there.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, Q-10, WCX-2, CM-10, IMAC-3, IMAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, Q-10, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 and Q-10 biochips have quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu⁺⁺ and Ni⁺⁺, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application US 2003 0032043 A1 (Pohl and Papanu, “Latex Based Adsorbent Chip,” Jul. 16, 2002) and U.S. patent application 60/350,110 (Um et al., “Hydrophobic Surface Chip,” Nov. 8, 2001); U.S. patent application 60/367,837, (Boschetti et al., “Biochips With Surfaces Coated With Polysaccharide-Based Hydrogels,” May 5, 2002) and U.S. patent application entitled “Photocrosslinked Hydrogel Surface Coatings” (Huang et al., filed Feb. 21, 2003).

Introduction

The present invention provides a polymeric analyte-adsorbing material that is appropriate for use, inter alia, in conjunction with desorption/ionization modes of mass spectrometric analysis. The properties of the material of the invention can be tuned by varying the structure of the monomers utilized in forming the polymer. For example, the concentration of luminescent moieties within the material can be varied to provide the appropriate density of luminescent moiety bonded (covalently or noncovalently). Moreover, the invention provides a polymeric material in which the luminescent moieties are combined with affinity reagents (“binding functionalities”), both chemical and/or biological, for the specific purpose of capturing (adsorbing) specific analyte molecules or classes of analyte molecules for the subsequent preparation, modification, and desorption of the analyte molecules.

A still further object is to provide a method for the quality control of an apparatus that is of use for desorption and ionization of analytes. The method is possible through the use of the luminescent polymer of the invention. The method includes interrogating the device using a method that detects luminescence to determine the distribution of luminescence originating from the luminescent polymer bound to the substrate.

The present invention is further explained and illustrated in the sections which follow, by reference to a representative embodiment using detection by mass spectrometry. The focus on mass spectrometric detection is for purposes of clarity and simplicity of illustration only, and should not be construed as limiting the scope of the present invention or circumscribing the types of methods in which the present invention finds application. Those of skill in the art will recognize that the methods set forth herein are broadly applicable to a number of chip formats and assays using these chips for the detection of a wide range of target moieties.

The Polymer

As discussed previously, the present invention provides a polymeric material that includes a luminescent moiety that is incorporated into the polymeric framework. Few structural restrictions are placed upon the luminescent moieties useful in practicing the present invention. The luminescent moiety can be an integral, covalently bonded component of the polymer, or it can be a species that is entrained within a polymeric matrix. When the luminescent moiety is not covalently bonded to the polymer it preferably interacts with the polymer via electrostatic, ionic, hydrophilic, or hydrophobic attraction. The luminescent moiety may also be entrained within the polymer by virtue of its being too large to diffuse from or otherwise exit the polymer.

The luminescent moieties of use in practicing the present invention will generally be based upon a homoaromatic or heteroaromatic nucleus. One of skill will appreciate that appropriate nuclei include monocylic (e.g., benzene, pyridine, pyrrole, furan, thiophene) as well as polycyclic systems. Moreover, when the aromatic nucleus is polycyclic, the ring system can be fused (e.g., naphthalene, benzofuran), or bonded in another fashion (e.g., biphenyl).

The aromatic nucleus is functionalized with a polymerizable reactive functional group. Those of skill will appreciate that an array of functional groups are appropriate for polymerizing monomers, and the present invention is not limited by the nature of the polymerizable functionality. Representative polymerizable reactive functional groups are set forth below.

Exemplary reactive functional groups (e.g., X¹ and X²) include: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups, which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups, which are capable of participating in Diels-Alder reactions such as, for example, maleimido groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides or reacted with acyl halides; (h) amine or sulfhydryl groups, which can be, for example, acylated or alkylated; (i) alkenes, which can undergo, for example, cycloadditions, acylation, Michael addition, etc; and (j) epoxides, which can react with, for example, amines and hydroxyl compounds.

The reactive functional groups can be chosen such that they do not participate in, or interfere with reactions in which they are not intended to participate. Alternatively, the reactive functional group can be protected from participating in the reaction by the presence of a protecting group. Those of skill in the art will understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

Those of skill in the art will understand that the reactive functional groups discussed herein represent only a subset of functional groups that are useful in assembling the matrix of the invention.

In a particularly preferred embodiment, the reactive functional group includes an unsaturated carbon-carbon or carbon-heteroatom bond. In a still further preferred embodiment, the reactive functional group includes at least one vinyl group, which is suitable for polymerization.

One skilled in the art will readily appreciate that many of these linkages may be produced in a variety of ways and using a variety of conditions. For the preparation of esters, see, e.g., March supra at 1157; for thioesters, see, March, supra at 362-363, 491, 720-722, 829, 941, and 1172; for carbonates, see, March, supra at 346-347; for carbamates, see, March, supra at 1156-57; for amides, see, March supra at 1152; for ureas and thioureas, see, March supra at 1174; for acetals and ketals, see, Greene et al. supra 178-210 and March supra at 1146; for acyloxyalkyl derivatives, see, PRODRUGS: TOPICAL AND OCULAR DRUG DELIVERY, K. B. Sloan, ed., Marcel Dekker, Inc., New York, 1992; for enol esters, see, March supra at 1160; for N-sulfonylimidates, see, Bundgaard et al., J. Med. Chem., 31:2066 (1988); for anhydrides, see, March supra at 355-56, 636-37, 990-91, and 1154; for N-acylamides, see, March supra at 379; for N-Mannich bases, see, March supra at 800-02, and 828; for hydroxymethyl ketone esters, see, Petracek et al. Annals NY Acad. Sci., 507:353-54 (1987); for disulfides, see, March supra at 1160; and for phosphonate esters and phosphonamidates, see, e.g., copending application Ser. No. 07/943,805, which is expressly incorporated herein by reference.

In the chips of the present invention, a class or type of molecular recognition event (e.g., adsorbent-target interaction) occurs at a binding functionality of an adsorbent film, thereby immobilizing a target on the film. The event is characterized by a particular selectivity condition, preferably occurring at an addressable location within the film.

In a preferred embodiment, the adsorbent film of the chips of the invention are configured such that detection of the immobilized analyte does not require elution, recovery, amplification, or labeling of the target analyte. Moreover, in another embodiment, the detection of one or more molecular recognition events, at one or more locations within the addressable adsorbent film, does not require removal or consumption of more than a small fraction of the total adsorbent-analyte complex. Thus, the unused portion can be interrogated further after one or more “secondary processing” events conducted directly in situ (i.e., within the boundary of the addressable location) for the purpose of structure and function elucidation, including further assembly or disassembly, modification, or amplification (directly or indirectly).

Adsorbents with improved specificity for an analyte can be developed by an iterative process, referred to as “progressive resolution,” in which adsorbents or eluants proven to retain an analyte are tested with additional variables to identity combinations with better binding characteristics. Another method allows the rapid creation of substrates with antibody adsorbents specific for an analyte. The method involves docking the analyte to an adsorbent, and screening phage display libraries for phage that bind the analyte.

The adsorbent film is attached to the linker arm layer by one of many interaction modalities with which one of skill in the art is familiar. Representative modalities include, but are not limited to, covalent attachment, attachment via polymer entanglement and electrostatic attachment. In a preferred embodiment, the film is immobilized onto the film support by electrostatic attraction.

Unlike covalent attachment and polymer entanglement methodologies, electrostatic attachment is virtually instantaneous and film thickness is independent of attachment conditions. Covalent attachment and polymer entanglement methodologies suffer from many of the process control variables mentioned previously with respect to polymer synthesis. As a result, neither of these attachment methodologies can achieve uniformity in film thickness that approaches what is achievable using electrostatic attachment. In an exemplary embodiment, electrostatic attachment is as simple as depositing a slurry of charged particles onto an oppositely charged surface, and then rinsing excess particles off the surface. Film thickness is defined by the diameter of the particles. Electrostatic repulsions between coating particles preferably prevents multiple layers of particles becoming attached to the surface.

As discussed in the preceding section, the linker is preferably a multiply charged polymer. Thus, in a further preferred embodiment, the adsorbent layer is a polymeric material having a net charge that is the opposite of the net charge of the linker. A complex is formed between the two oppositely charged materials, thereby immobilizing the adsorbent film onto the intermediate layer.

The adsorbent layer of the chip of the invention is preferably a polymeric material and it can include biological polymers, synthetic polymers, hybrids of biological and synthetic polymers and combinations of these polymers. The only limitation on the structure of a polymer useful in the chips of the invention is that the polymer adhere to, bond to or be otherwise immobilized upon the intermediate layer.

The following discussion sets forth representative methods of preparing adsorbent particles of use in preparing the adsorbent films of the chips of the invention. The discussion is intended to be illustrative and does not define or limit the scope of particle types that are useful in the present invention.

In a preferred embodiment, the particles are used as a latex, or other aqueous particle dispersion. Exemplary latex particles are disclosed in U.S. Pat. Nos. 4,101,460; 4,252,644; 4,351,909; 4,383,047; 4,519,905; 4,927,539; 5,324,752; and 5,532,279. The term “latex” is used loosely herein to include classic latexes, comprising particles whose diameter generally is less than 1 um, as well as small particles more formally defined as resins, having diameters less than 30 um.

Representation methods of preparing latex particles are known in the art. For example, a process for the preparation of finely particulate plastics dispersions from a monomer mixture comprising various ethylenically unsaturated monomers, including 0.1 to 5% by weight of an α-, β-unsaturated monocarboxylic acid is disclosed in U.S. Pat. No. 4,193,902. The process involves metering the monomer mix simultaneously with an initiator into an aqueous liquor containing from 0.5 to 10% by weight of an anionic emulsifier, polymerizing the monomers to form the dispersion, and adjusting the dispersion to a pH of 7 to 10.

Dispersions of polymers comprising an olefinically unsaturated dicarboxylic acid or anhydride are disclosed in U.S. Pat. No. 5,356,968. The dispersions are obtained by emulsion polymerization of the monomers in the presence of an emulsifier mixture comprising at least two anionic emulsifiers and optionally one or more nonionic emulsifiers.

In DE-A-4026640, it is disclosed that oligomeric carboxylic acids can be used as stabilizers for the emulsion polymerization of olefinically unsaturated monomers and that this leads to fine particulate polymer dispersions that are coagulate-free and extremely shear stable.

Aqueous coating and lacquer compositions comprising a graft copolymer having carboxylic-acid functional moieties attached at a terminal end thereof to a polymeric backbone are described in WO-A-9532228 and WO-A-95322255.

Known anion-exchange compositions generally fall into several categories. In the more traditional anion-exchange systems, synthetic support resin particles, generally carrying a negative charge, are covered with a layer of smaller synthetic resin particles carrying anion-exchange functional groups of positive charge, i.e. anion-exchange sites. The smaller particles are retained on the larger support particles via electrostatic attraction. The support resin can take a variety of forms. See, for example U.S. Pat. Nos. 4,101,460; 4,383,047; 4,252,644; 4,351,909; and 4,101,460.

A more recent development utilizes an uncharged support resin and smaller latex particles containing anion-exchange functional groups, held together by a dispersant. See, U.S. Pat. No. 5,324,752.

Monomers used to form the particle components of the adsorbent film are preferably selected from the group consisting of methyl(meth)acrylate, ethyl(meth)acrylate, butyl(meth)acrylate, 2-ethylhexyl(meth)acrylate, decyl(meth)acrylate, lauryl(meth)acrylate, isobornyl(meth)acrylate, isodecyl(meth)acrylate, oleyl(meth)acrylate, palmityl(meth)acrylate, steryl(meth)acrylate, styrene, butadiene, vinyl acetate, vinyl chloride, vinylidene chloride, vinylbenzyl chloride, vinylbenzyl glycidyl ether, acrylonitrile, methacrylonitrile, acrylamide and glycidylmethacrylate, hydroxyethyl(meth)acrylate, hydroxypropyl(meth)acrylate, acrylic acid, methacrylic acid, crotonic acid, fumaric acid, maleic acid, mono-methyl itaconate, mono-methyl fumarate, monobutyl fumarate, maleic anhydride, substituted acrylamides, diacetone acrylamide, acetoacetoxy ethyl methacrylate, acrolein, methacrolein, dicyclopentadienyl methacrylate, dimethyl meta-isopropenyl benzyl isocyanate, isocyanato ethyl methacrylate, methyl cellulose, hydroxyethyl cellulose, ethylene, propylene, N-vinyl pyrrolidone, and N,N′-dimethylamino(meth)acrylate.

In certain embodiments, it is preferred to crosslink a percentage of the particle components of the adsorbent film. Any cross-linking agent, useful to crosslink the particles can be used to prepare the chips of the invention. In a preferred embodiment, the crosslinking agent is a polymerizable monomer. Preferred addition polymerizable crosslinking precursors include: ethylene glycol dimethacrylate (EGDMA); ethylene glycol diacrylate (EGDA); propylene glycol dimethacrylate; propylene glycol diacrylate; butylene glycol dimethacrylate; butylene glycol diacrylate; hexamethylene glycol dimethacrylate; hexamethylene glycol diacrylate; pentamethylene glycol diacrylate; pentamethylene glycol dimethacrylate; decamethylene glycol diacrylate; decamethylene glycol dimethacrylate; vinyl acrylate; divinyl benzene; glycerol triacrylate; trimethylolpropane triacrylate; pentaerythritol triacrylate; polyoxyethylated trimethylolpropane triacrylate and trimethacrylate and similar compounds as disclosed in U.S. Pat. Nos. 3,380,831; 2,2-di(p-hydroxyphenyl)-propane diacrylate; pentaerythritol tetraacrylate; 2,2-di-(p-hydroxyphenyl)-propane dimethacrylate; triethylene glycol diacrylate; polyoxyethyl-2,2-di-(p-hydroxyphenyl)-propane dimethacrylate; di-(3-methacryloxy-2-hydroxypropyl)ether of bisphenol-A; di-(2-methacryloxyethyl)ether of bisphenol-A; di-(3-acryloxy-2-hydroxypropyl)ether of bisphenol-A; di-(2-acryloxyethyl)ether of bisphenol-A; di-(3-methacryloxy-2-hydroxypropyl)ether of tetrachloro-bisphenol-A; di-(2-methacryloxyethyl)ether of tetrachloro-bisphenol-A; di-(3-methacryloxy-2-hydroxypropyl)ether of tetrabromo-bisphenol-A; di-(2-methacryloxyethyl)ether of tetrabromo-bisphenol-A; di-(3-methacryloxy-2-hydroxypropyl)ether of 1,4-butanediol; di-(3-methacryloxy-2-hydroxypropyl)ether of diphenolic acid; triethylene glycol dimethacrylate; polyoxypropyl one trimethylol propane triacrylate (462); 1,2,4-butanetriol trimethacrylate; 2,2,4-trimethyl-1,3-pentanediol dimethacrylate; pentaerythritol trimethacrylate; 1-phenyl ethylene-1,2-dimethacrylate; pentaerythritol tetramethacrylate; trimethylol propane trimethacrylate; 1,5-pentanediol dimethacrylate; diallyl fumarate; 1,4-benzenediol dimethacrylate; 1,4-diisopropenyl benzene; and 1,3,5-triisopropenyl benzene. A class of addition polymerizable crosslinking precursors are an alkylene or a polyalkylene glycol diacrylate or dimethacrylate prepared from an alkylene glycol of 2 to 15 carbons or a polyalkylene ether glycol of 1 to 10 ether linkages, and those disclosed in U.S. Pat. No. 2,927,022, e.g., those having a plurality of addition polymerizable ethylenic linkages particularly when present as terminal linkages. Members of this class are those wherein at least one and preferably most of such linkages are conjugated with a double bonded carbon, including carbon double bonded to carbon and to such heteroatoms as nitrogen, oxygen and sulfur. Also included are such materials wherein the ethylenically unsaturated groups, especially the vinylidene groups, are conjugated with ester or amide structures and the like.

The polymer of the invention can also include a binding functionality for an analyte within its polymeric structure. For purposes of convenience, both the binding functionality and components of the binding functionality are referred to as the binding functionality. The binding functionality is selected from an electrostatic functionality, a hydrophobic functionality, a hydrogen bonding functionality, a coordinate covalent bonding functionality, a covalent bonding functionality, a biospecific bonding functionality and combinations thereof.

In an exemplary embodiment, the binding functionality comprises an organic functional group that interacts with a component of the analyte. In an exemplary embodiment, the organic functional group is selected from simple groups, such as amines, carboxylic acids, sulfonic acids, alcohols, sulfhydryls and the like. Functional groups presented by more complex species are also of use, such as those presented by drugs, chelating agents, crown ethers, cyclodextrins, and the like. In an exemplary embodiment, the binding functionality is an amine that interacts with a structure on the analyte that binds to the amine (e.g., carbonyl groups, alkylhalo groups), or which protonates the amine (e.g., carboxylic acid, sulfonic acid) to form an ion pair. In another exemplary embodiment, the binding functionality is a carboxylic acid, which interacts with the analyte by complexation (e.g., metal ions), or which protonate a basic group on the analyte (e.g. amine) forming an ion pair.

The organic functional group can also be a component of a small organic molecule with the ability to specifically recognize an analyte molecule. Exemplary small organic molecules include, but are not limited to, amino acids, biotins, carbohydrates, glutathiones, and nucleic acids.

Exemplary amino acids suitable as binding functionalities, include L-alanine, L-arginine, L-asparagine, L-aspartic acid, L-cysteine, L-cystine, L-glutamic acid, L-glutamine, glycine, L-histidine, L-isoleucine, L-lysine, L-methionine, L-phenylalanine, L-proline, L-serine, L-threonine, L-thyroxine, D-tryptophan, L-tryptophan, L-tyrosine and L-valine. Typical avidin-biotin ligands include avidin, biotin, desthiobiotin, diaminobiotin, and 2-iminobiotin. Typical carbohydrates include glucoseamines, glycopryranoses, galactoseamines, the fucosamines, the fucopyranosylamines, the galactosylamines, the glycopyranosides, and the like. Typical glutathione ligands include glutathione, hexylglutathione, and sulfobromophthalein-S-glutathione.

In another exemplary embodiment, the binding functionality is a biomolecule, e.g., a natural or synthetic peptide, antibody, nucleic acid, saccharide, lectin, receptor, antigen, cell or a combination thereof. Thus, in an exemplary embodiment, the binding functionality is an antibody raised against an analyte or against a species that is structurally analogous to an analyte. In another exemplary embodiment, the binding functionality is avidin, or a derivative thereof, which binds to a biotinylated analogue of the analyte. In still another exemplary embodiment, the binding functionality is a nucleic acid, which binds to single- or double-stranded nucleic acid analyte having a sequence complementary to that of the binding functionality.

Biomolecules useful in practicing the present invention are derived from any source. The biomolecules can be isolated from natural sources or they can be produced by synthetic methods. Proteins can be natural proteins, mutated proteins or fusion proteins. Mutations can be effected by chemical mutagenesis, site-directed mutagenesis or other means of inducing mutations known to those of skill in the art. Proteins useful in practicing the instant invention include, for example, enzymes, antigens, antibodies and receptors. Antibodies can be either polyclonal or monoclonal.

Binding functionalities, which are antibodies can be used to recognize analytes which include, but are not limited to, proteins, peptides, nucleic acids, saccharides or small molecules such as drugs, herbicides, pesticides, industrial chemicals, organisms, cells and agents of war. Methods of raising antibodies against specific molecules or organisms are well known to those of skill in the art. See, U.S. Pat. No. 5/147,786, issued to Feng et al. on Sep. 15, 1992; No. 5/334,528, issued to Stanker et al. on Aug. 2, 1994; No. 5/686,237, issued to Al-Bayati, M.A.S. on Nov. 11, 1997; and No. 5/573,922, issued to Hoess et al. on Nov. 12, 1996.

Antibodies and other peptides can be attached to the adsorbent film by any known method. For example, peptides can be attached through an amine, carboxyl, sulfhydryl, or hydroxyl group. The site of attachment can reside at a peptide terminus or at a site internal to the peptide chain. The peptide chains can be further derivatized at one or more sites to allow for the attachment of appropriate reactive groups onto the chain. See, Chrisey et al. Nucleic Acids Res. 24:3031-3039 (1996). Methods for attaching antibodies to surfaces are also known in the art. See, Delamarche et al. Langmuir 12:1944-1946 (1996).

In another exemplary embodiment, the chip of this invention is an oligonucleotide array in which the binding functionality at each addressable location in the array comprises a nucleic acid having a particular nucleotide sequence. In particular, the array can comprise oligonucleotides. For example, the oligonucleotides could be selected so as to cover the sequence of a particular gene of interest. Alternatively, the array can comprise cDNA or EST sequences useful for expression profiling.

In another exemplary embodiment, the binding functionality is a drug moiety or a pharmacophore derived from a drug moiety. The drug moieties can be agents already accepted for clinical use or they can be drugs whose use is experimental, or whose activity or mechanism of action is under investigation. The drug moieties can have a proven action in a given disease state or can be only hypothesized to show desirable action in a given disease state. In a preferred embodiment, the drug moieties are compounds, which are being screened for their ability to interact with an analyte of choice. As such, drug moieties, which are useful in practicing the instant invention include drugs from a broad range of drug classes having a variety of pharmacological activities.

When the binding functionality is a chelating agent, crown ether or cyclodextrin, host-guest chemistry will dominate the interaction between the binding functionality and the analyte. The use of host-guest chemistry allows a great degree of affinity-moiety-analyte specificity to be engineered into a device of the invention. The use of these compounds to bind to specific compounds is well known to those of skill in the art. See, for example, Pitt et al. “The Design of Chelating Agents for the Treatment of Iron Overload,” In, INORGANIC CHEMISTRY IN BIOLOGY AND MEDICINE; Martell, A. E., Ed.; American Chemical Society, Washington, D.C., 1980, pp. 279-312; Lindoy, L. F., THE CHEMISTRY OF MACROCYCLIC LIGAND COMPLEXES; Cambridge University Press, Cambridge, 1989; Dugas, H., BIOORGANIC CHEMISTRY; Springer-Verlag, New York, 1989, and references contained therein.

Additionally, a number of routes allowing the attachment of chelating agents, crown ethers and cyclodextrins to other molecules are available to those of skill in the art. See, for example, Meares et al., “Properties of In vivo Chelate-Tagged Proteins and Polypeptides.” In, MODIFICATION OF PROTEINS: FOOD, NUTRITIONAL, AND PHARMACOLOGICAL ASPECTS;” Feeney, R. E., Whitaker, J. R., Eds., American Chemical Society, Washington, D.C., 1982, pp. 370-387; Kasina et al. Bioconjugate Chem. 9:108-117 (1998); Song et al., Bioconjugate Chem. 8:249-255 (1997).

In an exemplary embodiment, the binding functionality is a polyaminocarboxylate chelating agent such as ethylenediaminetetraacetic acid (EDTA) or diethylenetriaminepentaacetic acid (DTPA), which is attached to an amine on the substrate, or spacer arm, by utilizing the commercially available dianhydride (Aldrich Chemical Co., Milwaukee, Wis.). When complexed with a metal ion, the metal chelate binds to tagged species, such as polyhistidyl-tagged proteins, which can be used to recognize and bind analyte species. Alternatively, the metal ion itself, or a species complexing the metal ion can be the analyte.

In a further exemplary embodiment, the binding functionality forms an inclusion complex with the analyte of interest. In a preferred embodiment, the binding functionality is a cyclodextrin or modified cyclodextrin. Cyclodextrins are a group of cyclic oligosaccharides produced by numerous microorganisms. Cyclodextrins have a ring structure which has a basket-like shape. This shape allows cyclodextrins to include many kinds of molecules into their internal cavity. See, for example, Szejtli, J., CYCLODEXTRINS AND THEIR INCLUSION COMPLEXES; Akademiai Klado, Budapest, 1982; and Bender et al., CYCLODEXTRIN CHEMISTRY, Springer-Verlag, Berlin, 1978. Cyclodextrins are able to form inclusion complexes with an array of organic molecules including, for example, drugs, pesticides, herbicides and agents of war. See, Tenjarla et al., J. Pharm. Sci. 87:425-429 (1998); Zughul et al., Pharm. Dev. Technol. 3:43-53 (1998); and Albers et al., Crit. Rev. Ther. Drug Carrier Syst. 12:311-337 (1995). Importantly, cyclodextrins are able to discriminate between enantiomers of compounds in their inclusion complexes. Thus, in one preferred embodiment, the invention provides for the detection of a particular enantiomer in a mixture of enantiomers. See, Koppenhoefer et al. J. Chromatogr. A 793:153-164 (1998). The cyclodextrin binding functionality can be attached to a spacer arm or directly to the substrate. See, Yamamoto et al., J. Phys. Chem. B 101:6855-6860 (1997). Methods to attach cyclodextrins to other molecules are well known to those of skill in the chromatographic and pharmaceutical arts. See, Sreenivasan, K. J. Appl. Polym. Sci. 60:2245-2249 (1996).

In a further preferred embodiment, the binding functionality is selected from nucleic acid species, such as aptamers and aptazymes that recognize specific analytes.

The Luminescent Moiety

The present invention provides a luminescent polymer that includes trace amounts of a luminescent moiety (“lumophore”) within the polymeric framework. The luminescent moiety has a structure that is different from the structure of the moiety derived from the predominant monomer (“bulk monomer”) that is used to assemble the polymer. Thus, although the bulk monomer may have intrinsic luminescent properties, the luminescence produced by the luminescent monomer is distinguishable from that produced by the bulk monomer. The basis for the distinction is, for example, a difference in quantum yields and, consequently a difference in measured luminescence intensities, different absorbance wavelengths, and different emission wavelengths. The luminescence produced by the luminescent moiety is resolved over the background of the bulk polymer. Similarly, if the substrate has inherent luminescent properties in the absence of the luminescent moiety, the luminescence produced by the luminescent moiety is resolved over that produced by the substrate.

In a presently preferred embodiment, the lumiphore has a fluorescence quantum yield of greater than 0.2, more preferably, greater than or equal to about 0.25 and more preferably still greater than or equal to about 0.35 at 25° C. In another preferred embodiment, the luminescent moiety of the bulk monomer is less than or equal to about 0.2, more preferably, less than about 0.15 and even more preferably, less than about 0.1.

Luminescent labels have the advantage of requiring few precautions in their handling, and being amenable to high-throughput visualization techniques (optical analysis including digitization of the image for analysis in an integrated system comprising a computer). Preferred labels are typically characterized by one or more of the following: high sensitivity, high stability, low background, long lifetimes, low environmental sensitivity and high specificity in labeling.

Many fluorophores useful in practicing the present invention are commercially available from, for example, the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophore de novo or synthetically modify commercially available fluorescent compounds to arrive at the desired fluorescent label.

A non-limiting list of luminescent groups that can be used in conjunction with the invention is provided in Table 1.

TABLE 1
Representative Luminescent Groups

4-acetamido-4′-isothiocyanatostilbene-2,2′disulfonic acid
acridine and derivatives:

acridine
acridine isothiocyanate

5-(2′-aminoethyl)aminonaphthalene-1-sulfonic acid (EDANS)
4-amino-N-[3-vinylsulfonyl)phenyl]naphthalimide-3,5 disulfonate
N-(4-anilino-1-naphthyl)maleimide
anthranilamide
BODIPY
Brilliant Yellow
coumarin and derivatives:
coumarin

7-amino-4-methylcoumarin (AMC, Coumarin 120)
7-amino-4-trifluoromethylcouluarin (Coumaran 151)

cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin and derivatives:

eosin
eosin isothiocyanate

erythrosin and derivatives:

erythrosin B
erythrosin isothiocyanate

ethidium
fluorescein and derivatives:

5-carboxyfluorescein (FAM)
5-(4,6-dichlorotriazin-2-yl)aminofluorescein (DTAF)
2′,7′-dimethoxy-4′5′-dichloro-6-carboxyfluorescein (JOE)
fluorescein
fluorescein isothiocyanate
QFITC (XRITC)

fluorescamine
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
pyrene and derivatives:

pyrene
pyrene butyrate
succinimidyl 1-pyrene butyrate

quantum dots
anthracene and derivatives
Reactive Red 4 (Cibacron ™ Brilliant Red 3B-A)
rhodamine and derivatives:

6-carboxy-X-rhodamine (ROX)
6-carboxyrhodamine (R6G)
lissamine rhodamine B sulfonyl chloride rhodamine (Rhod)
rhodamine B
rhodamine 123
rhodamine X isothiocyanate
sulforhodamine B
sulforhodamine 101

sulfonyl chloride derivative of sulforhodamine 101 (Texas Red)
N,N,N′,N′-tetramethyl-6-carboxyrhodamine (TAMRA)
tetramethyl rhodamine

tetramethyl rhodamine isothiocyanate (TRITC)

riboflavin
rosolic acid
lanthanide chelate derivatives

There is a great deal of practical guidance available in the literature for selecting appropriate donor-acceptor pairs for particular probes, as exemplified by the following references: Pesce et al., Eds., FLUORESCENCE SPECTROSCOPY (Marcel Dekker, New York, 1971); White et al., FLUORESCENCE ANALYSIS: A PRACTICAL APPROACH (Marcel Dekker, New York, 1970); and the like. The literature also includes references providing exhaustive lists of fluorescent and chromogenic molecules and their relevant optical properties, for choosing reporter-quencher pairs (see, for example, Berlman, HANDBOOK OF FLUORESCENCE SPECTRA OF AROMATIC MOLECULES, 2nd Edition (Academic Press, New York, 1971); Griffiths, COLOUR AND CONSTITUTION OF ORGANIC MOLECULES (Academic Press, New York, 1976); Bishop, Ed., INDICATORS (Pergamon Press, Oxford, 1972); Haugland, HANDBOOK OF FLUORESCENT PROBES AND RESEARCH CHEMICALS (Molecular Probes, Eugene, 1992) Pringsheim, FLUORESCENCE AND PHOSPHORESCENCE (Interscience Publishers, New York, 1949); and the like. Further, there is extensive guidance in the literature for derivatizing reporter and quencher molecules for covalent attachment via readily available reactive groups that can be added to a molecule.

Those of skill will readily appreciate that the luminescent groups can be modified to bear a selected reactive group that provides for their incorporation into the polymer of the invention. The diversity and utility of chemistries available for conjugating fluorophores to other molecules is exemplified by the extensive body of literature on preparing nucleic acids derivatized with fluorophores. See, for example, Haugland (supra); Ullman et al., U.S. Pat. No. 3,996,345; Khanna et al., U.S. Pat. No. 4,351,760. Thus, it is well within the abilities of those of skill in the art to choose a polymerizable monomer with a reactive group that provides a locus for attaching the luminescent group to the polymerizable monomer.

In addition to small molecule fluorophores, naturally occurring fluorescent proteins and engineered analogues of such proteins are useful with the polymers of the present invention. Such proteins include, for example, green fluorescent proteins of cnidarians (Ward et al., Photochem. Photobiol. 35:803-808 (1982); Levine et al., Comp. Biochem. Physiol., 72B:77-85 (1982)), yellow fluorescent protein from Vibrio fischeri strain (Baldwin et al., Biochemistry 29:5509-15 (1990)), Peridinin-chlorophyll from the dinoflagellate Symbiodinium sp. (Morris et al., Plant Molecular Biology 24:673:77 (1994)), phycobiliproteins from marine cyanobacteria, such as Synechococcus, e.g., phycoerythrin and phycocyanin (Wilbanks et al., J. Biol. Chem. 268:1226-35 (1993)), and the like.

Preparation of the Polymer

In its most general aspect, the preparation of the polymer involves the selection of an appropriate polymerizable luminescent monomer and the co-polymerization of that monomer with a bulk monomer to form a luminescent co-polymer. The polymerizable luminescent monomer is either a commercially available monomer or it is prepared according to methods readily accessible to those of skill in the art. The polymerizable luminescent monomer generally comprises a luminescent moiety (“lumophore”) and a polymerizable moiety. Polymerizable moieties are well known in the art and include, for example vinyl, acryloyl, allyl groups and their derivatives.

By way of example, a representative scheme leading to the preparation of a luminescent polymer of the invention is set forth in Scheme 1.

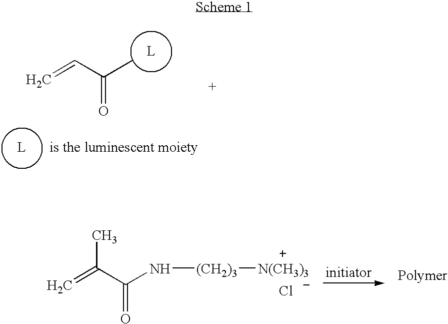

In Scheme 1, the polymerizable luminescent monomer includes an acryloyl moiety. The luminescent monomer is combined with a second complementary polymerizable monomer (“bulk monomer”) and a polymerization initiator. The polymer formed in exemplary Scheme 1 includes both a luminescent moiety and a binding moiety, i.e., an anion exchange moiety.

In an exemplary embodiment, the luminescent monomer is present in trace amounts relative to the other monomer(s). “Trace amount” as used herein generally means that the molar ratio of luminescent monomer to the other monomer(s) is typically from about 1:500 to about 1:150,000, preferably from about 1:1000 to about 1:100,000, even more preferably from about 1:5000 to about 1:20,000. An exemplary polymer of the invention has a molar ratio of luminescent monomer to the other monomer(s) of about 1:500,000. In parts per million, the luminescent monomer is preferably present between 2 ppm and 12 ppm total monomer species, more preferably around 7.5 ppm.

In another exemplary embodiment, the polymer includes the subunit: 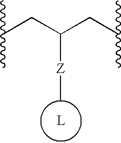
in which Z is a linker moiety that is selected from substituted or unsubstituted alkyl, substituted or unsubstituted heteroalkyl, and substituted or unsubstituted aryl moieties. The circle with the inset letter “L” represents a lumophore as discussed above. An exemplary luminescent monomer of use in the present invention is 9-anthracenylmethyl acrylate having the following formula:

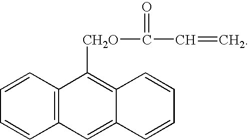

In a still further exemplary embodiment, the polymer of the invention includes the subunit:

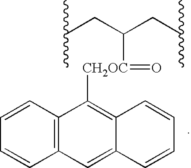

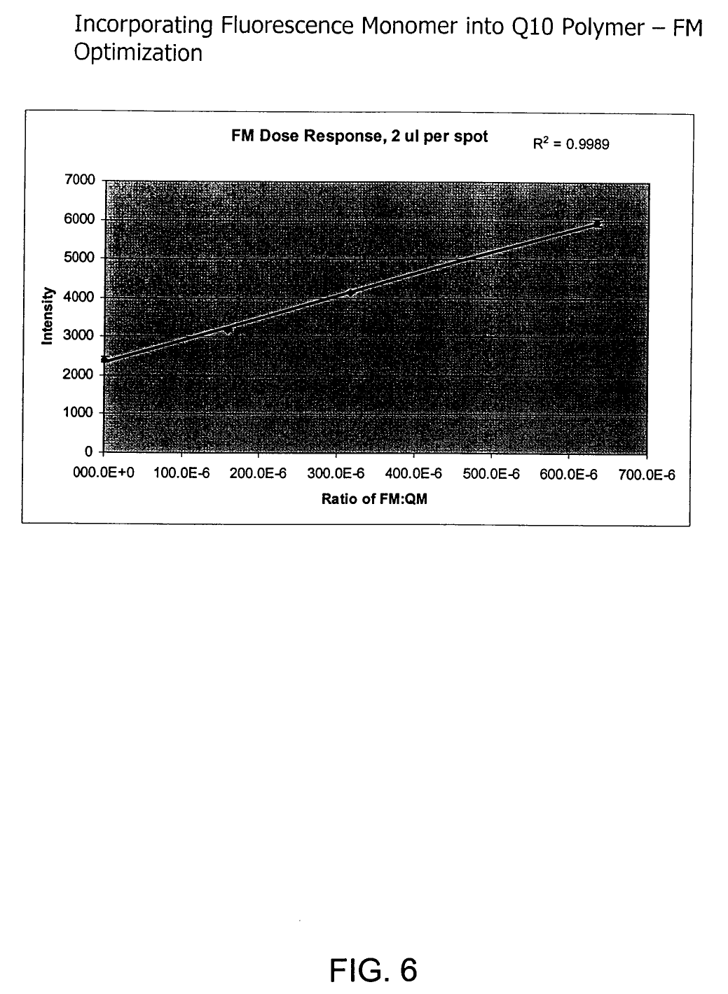

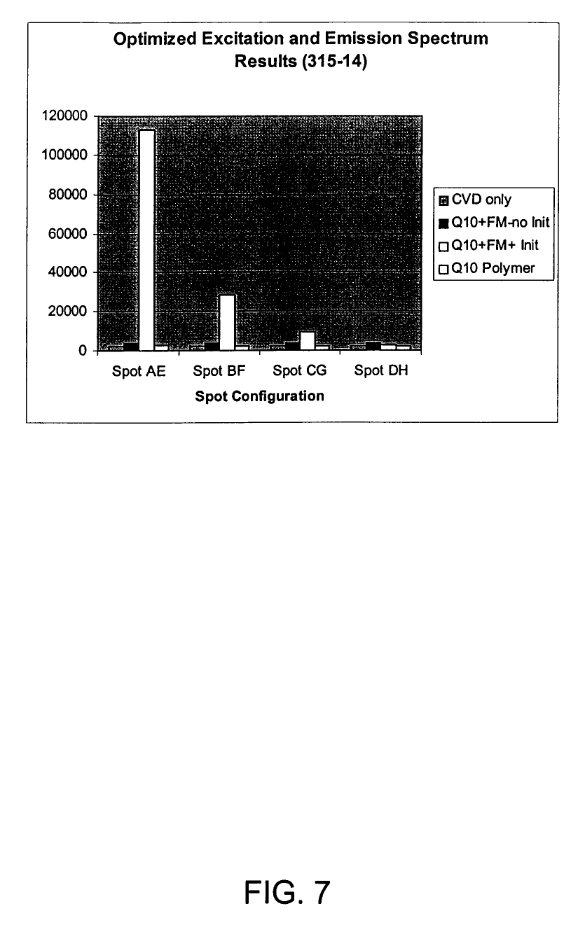

As shown in FIG. 6, the fluorescence intensity of the polymer increases in an essentially linear manner with increasing fluorescent monomer incorporation into the polymer. The fluorescence intensity of the polymer is sufficient to resolve it over background fluorescence from an exemplary chip substrate, and a polymer composed solely of the “bulk monomer”. Also the polymer of the invention is clearly resolved over a mixture of Q10 and the fluorescent monomer prepared in the absence of a polymerization initiator. See, FIG. 7.

In an exemplary preparation, the polymerization is initiated by the addition of a peroxide, such as lauroyl peroxide. The polymer is purified by methods known in the art, e.g., extraction of unreacted monomers, precipitation, crystallization, fractional crystallization, size exclusion chromatography, dialysis and the like. The polymer is also characterized by art-recognized methods, e.g., NMR, IR, size exclusion chromatography, elemental analysis and the like.

The efficacy of the polymers of the invention in an analysis can be assessed by applying a standard or known sample, e.g., peptide, nucleic acid, to the chip incorporating a polymer of the invention and performing the desired analysis. In an exemplary embodiment, a standard peptide solution is applied to the chip and a desorption/ionization analysis is performed.

Surprisingly, use of the polymers of the invention in conjunction with a desorption/ionization mass spectrometric analysis produces results that are essentially identical to those achieved using polymers that are not functionalized with a luminescent moiety.

The above scheme is offered to exemplify the general concept of preparing the polymeric material of the invention. Those of skill will appreciate that the polymeric compounds of the invention can be formed by any art-recognized method for polymerizing or copolymerizing monomers. The polymerization process can be accomplished using a number of possible synthetic routes including, but not limited to, homogeneous or heterogeneous chain-growth polymerization including a free radical or ionic polymerization reaction and photopolymerization with a photoinitiator, and step-growth polymerization, including addition-elimination reactions, addition-substitution reactions, nucleophilic substitution reactions, multiple-bond addition reactions, etc. The compositions of the invention can be prepared using bulk polymerization, solution polymerization, emulsion polymerization, suspension polymerization, condensation polymerization, etc. Suitable monomers are dependent upon the type of polymerization being utilized, and it is within the abilities of one of skill in the art to select the proper monomer and polymerization conditions to achieve a desired property or result.

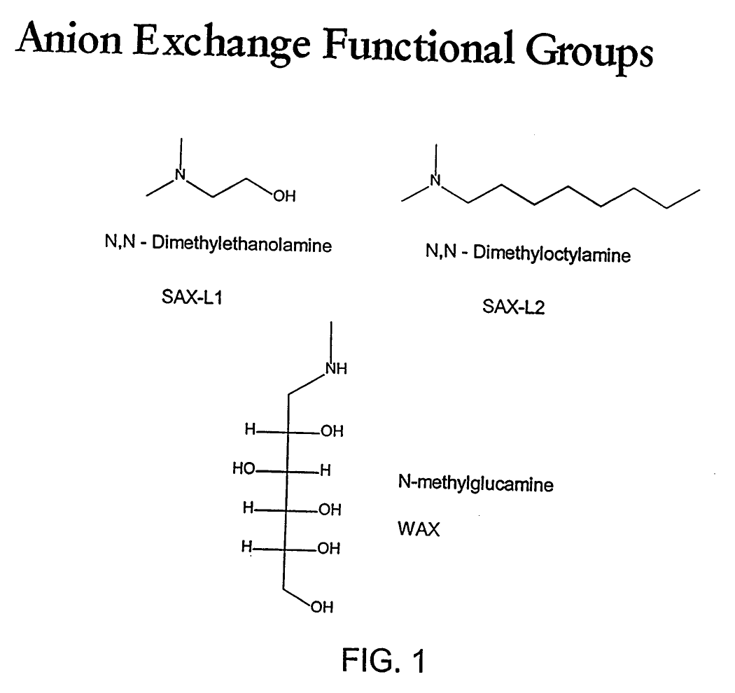

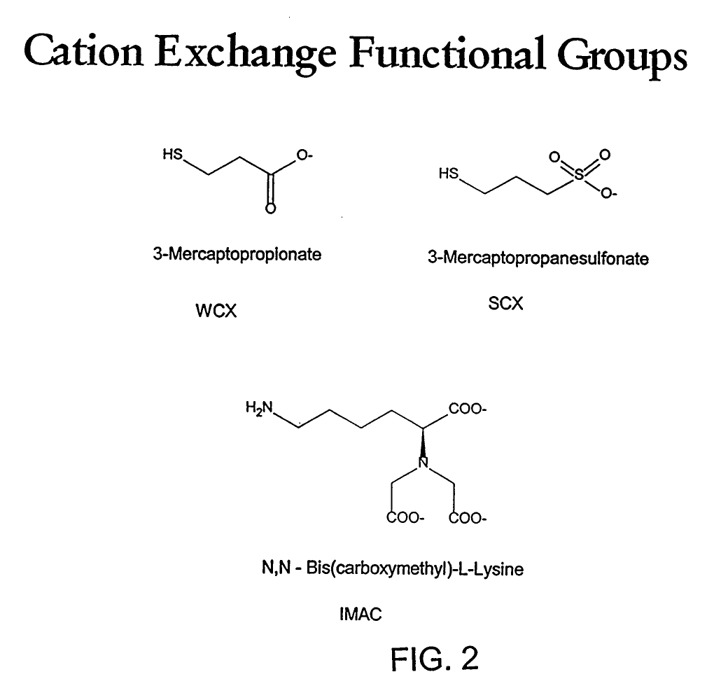

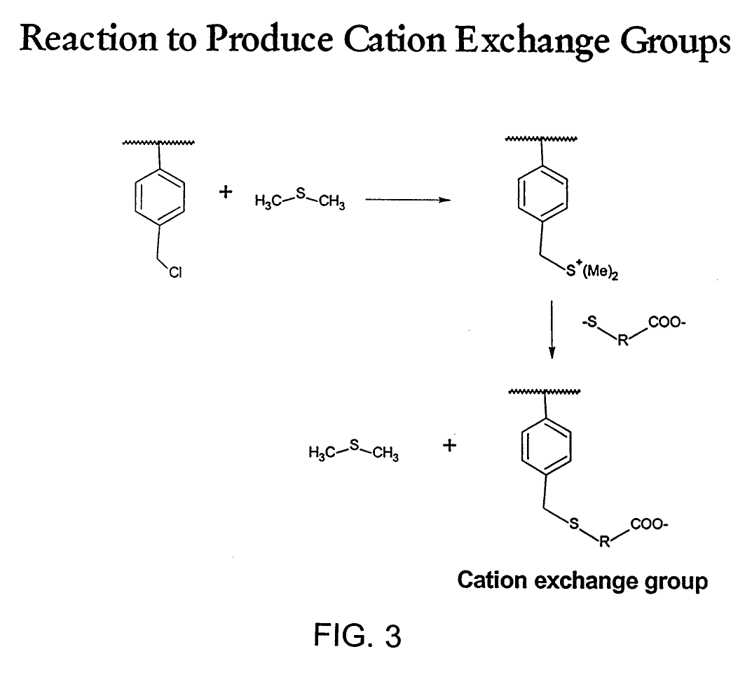

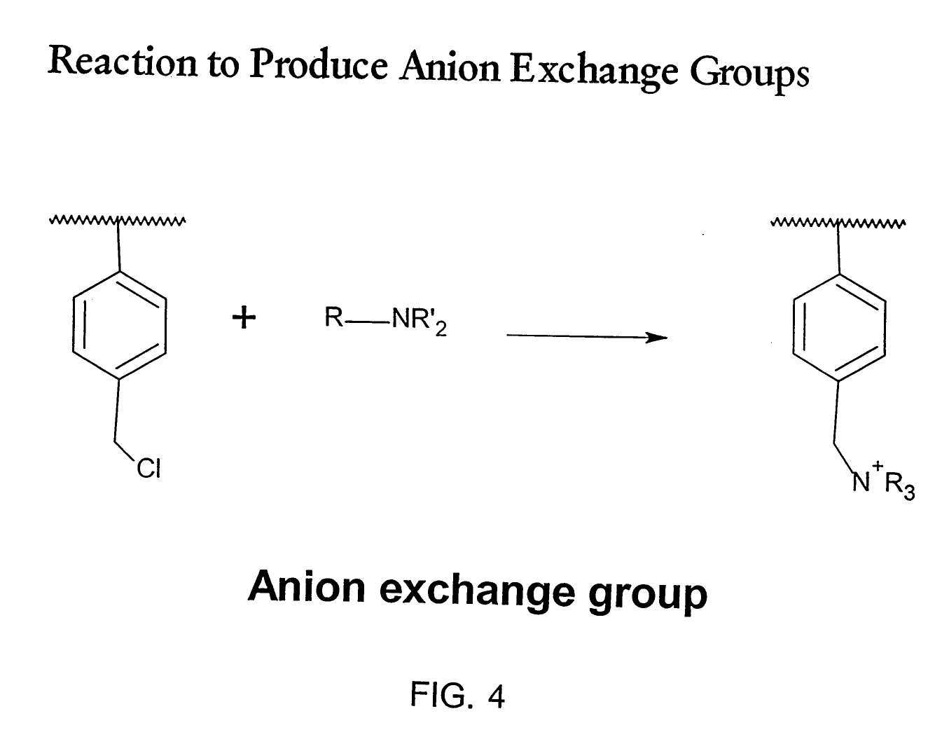

After synthesis, the monomers and/or completed polymer can be further elaborated by a variety of chemical reactions well known to those skilled in the art. For example, in order to produce a matrix with anion exchange properties, the luminescent monomer can be co-polymerized with monomer having primary, secondary, tertiary, quaternary amine or chloromethyl group which can be aminated and quaternized (FIG. 1 and FIG. 4). Production of an analogous polymeric compound, containing cation exchange sites can be accomplished by a number of well-known synthetic schemes. The luminescent monomer can be co-polymerized with monomers that include groups for cation exchange, such as sulfonic acid or carboxylic acid groups. See, FIG. 2 and FIG. 3.

In another exemplary embodiment, non-luminescent monomers can be a styrene monomer and the copolymer can be further aminated through chloromethylation or sulfonation or carboxylation to have ionic polymeric compounds. Also, a further representative method relies on the use of a dimethyl sulfide displacement reaction, in which a vinylbenzyl chloride-containing matrix component is first reacted with a solution of dimethyl sulfide (FIG. 3). The resulting reaction product is a sulfonium based anion exchange compound. A second cation exchange site generation reagent is then added to the reaction mixture, which can be heated in order to help drive the reaction to completion. An exemplary reagent for this purpose is mercaptopropionic acid. A solution of this acid is first pH adjusted to about 11 and then mixed with the above suspension of sulfonium based anion exchange matrix. After heating the suspension at about 70° C. for a predetermined period of time, the substitution reaction is complete and the resulting adsorbent film component is now a weak acid cation exchange matrix.

Similar reaction pathways are available for preparing polymeric compounds and polymeric components with other binding functionalities. It is within the abilities of one of skill in the art to determine an appropriate reaction pathway to conjugate a selected binding functionality to the polymeric compounds or polymeric components (see, for example, Hermanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991.

The Chip

Also provided by the instant invention are analytical devices that incorporate the polymeric compound of the invention. The present invention contemplates a range of analytical devices that incorporate the polymeric compound of the invention. The use of the polymeric compound in an analytical device is exemplified herein by reference to a chip, which is of use as a substrate or probe component in desorption/ionization mass spectrometric methods of analysis. The focus of the following discussion on the chip format and its use in mass spectrometric analyses is for clarity of illustration alone and is not intended to limit the scope of the invention.

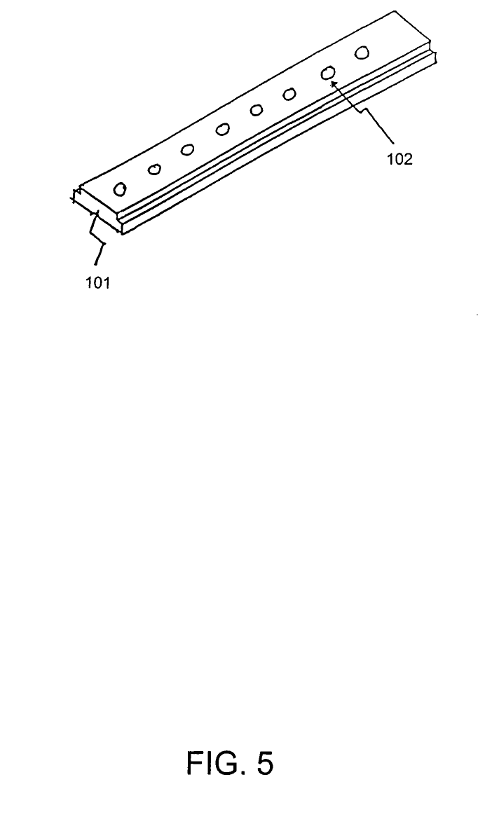

Thus, in one aspect, the present invention provides a device that includes a substrate having a surface, and a polymeric material of the invention attached to the surface. The polymeric material is adapted to receive analyte molecules and is of use in various detection and characterization methodologies. An exemplary chip format is set forth in FIG. 5.

The polymeric compound of the invention is generally anchored to the surface of the chip substrate. The interaction between the polymeric compound and the surface, which anchors the polymeric compound to the surface can be a covalent, electrostatic, ionic, hydrogen bonding, hydrophobic-hydrophobic, or hydrophilic-hydrophilic interaction. When the interaction is non-covalent, it is referred to herein as “physical adhesion.” The polymer can be anchored directly onto the substrate or, alternatively, it can be bound to the substrate through an “intermediate layer” that is itself bound to the substrate. In an exemplary embodiment of the “intermediate layer” motif, the polymer is a charged polymer and it is anchored to the substrate through interaction with an oppositely charged “intermediate layer.” Examples of devices of this configuration are discussed in copending, commonly assigned U.S. patent application Ser. No. 10/197,115, filed on Jul. 16, 2002. In another embodiment, the polymer is copolymerized with a reactive functional group on an intermediate layer that is anchored to the substrate. See, for example, copending, commonly assigned U.S. patent application Ser. No. 09/560,715, filed on Apr. 27, 2000. In yet another embodiment, the polymer has mixed hydrophilic and hydrophobic domains as set forth in copending PCT/US02/36171, filed on Nov. 7, 2002.

The Substrate

In the chip of the invention, the polymer is immobilized on a substrate, either directly or through linker arms that are intercalated between the substrate and the Polymer. The Polymer is immobilized on the plane of the substrate surface, or it is bound to a feature of the substrate surface, which may be flush with the surface, raised (e.g., island) or depressed (e.g., a well, trough, etc.). Substrates that are useful in practicing the present invention can be made of any stable material, or combination of materials. Moreover, useful substrates can be configured to have any convenient geometry or combination of structural features. The substrates can be either rigid or flexible and can be either optically transparent or optically opaque. The substrates can also be electrical insulators, conductors or semiconductors. When the sample to be applied to the chip is water based, the substrate preferable is water insoluble.

The materials forming the substrate are utilized in a variety of physical forms such as films, supported powders, glasses, crystals and the like. For example, a substrate can consist of a single inorganic oxide or a composite of more than one inorganic oxide. When more than one component is used to form a substrate, the components can be assembled in, for example a layered structure (i.e., a second oxide deposited on a first oxide) or two or more components can be arranged in a contiguous non-layered structure. Further the substrates can be substantially impermeable to liquids, vapors and/or gases or, alternatively, the substrates can be permeable to one or more of these classes of materials. Moreover, one or more components can be admixed as particles of various sizes and deposited on a support, such as a glass, quartz or metal sheet. Further, a layer of one or more components can be intercalated between two other substrate layers (e.g., metal-oxide-metal, metal-oxide-crystal). Those of skill in the art are able to select an appropriately configured substrate, manufactured from an appropriate material for a particular application.

Exemplary substrate materials include, but are not limited to, inorganic crystals, inorganic glasses, inorganic oxides, metals, organic polymers and combinations thereof. Inorganic glasses and crystals of use in the substrate include, but are not limited to, LiF, NaF, NaCl, KBr, KI, CaF₂, MgF₂, HgF₂, BN, AsS₃, ZnS, Si₃N₄, AIN and the like. The crystals and glasses can be prepared by art standard techniques. See, for example, Goodman, CRYSTAL GROWTH THEORY AND TECHNIQUES, Plenum Press, New York 1974. Alternatively, the crystals can be purchased commercially (e.g., Fischer Scientific). Inorganic oxides of use in the present invention include, but are not limited to, Cs₂O, Mg(OH)₂, TiO₂, ZrO₂, CeO₂, Y₂O₃, Cr₂O₃, Fe₂O₃, NiO, ZnO, Al₂O₃, SiO₂ (glass), quartz, In₂O₃, SnO₂, PbO₂ and the like. Metals of use in the substrates of the invention include, but are not limited to, gold, silver, platinum, palladium, nickel, copper and alloys and composites of these metals.

Metals are also of use as substrates in the present invention. The metal can be used as a crystal, a sheet or a powder. In those embodiments in which the metal is layered with another substrate component, the metal can be deposited onto the other substrate by any method known to those of skill in the art including, but not limited to, evaporative deposition, sputtering and electroless deposition.

The metal layers can be either permeable or impermeable to materials such as liquids, solutions, vapors and gases. Presently preferred metals include, but are not limited to, gold, silver, platinum, palladium, nickel, aluminum, copper, stainless steel, and other iron alloys.

Organic polymers that form useful substrates include, for example, polyalkenes (e.g., polyethylene, polyisobutene, polybutadiene), polyacrylics (e.g., polyacrylate, polymethyl methacrylate, polycyanoacrylate), polyvinyls (e.g., polyvinyl alcohol, polyvinyl acetate, polyvinyl butyral, polyvinyl chloride), polystyrenes, polycarbonates, polyesters, polyurethanes, polyamides, polyimides, polysulfone, polysiloxanes, polyheterocycles, cellulose derivative (e.g., methyl cellulose, cellulose acetate, nitrocellulose), polysilanes, fluorinated polymers, epoxies, polyethers and phenolic resins.

In a preferred embodiment, the substrate material is substantially non-reactive with the analyte, thus preventing non-specific binding between the substrate and the analyte or other components of an assay mixture. Methods of coating substrates with materials to prevent non-specific binding are generally known in the art. Exemplary coating agents include, but are not limited to cellulose, bovine serum albumin, and poly(ethyleneglycol). The proper coating agent for a particular application will be apparent to one of skill in the art.

In a further preferred embodiment, the substrate material is substantially non-fluorescent or emits light of a wavelength range that does not interfere with the detection of the analyte. Exemplary low-background substrates include those disclosed by Cassin et al, U.S. Pat. No. 5,910,287 and Pham et al., U.S. Pat. No. 6,063,338.

The surface of a substrate of use in practicing the present invention can be smooth, rough and/or patterned. The surface can be engineered by the use of mechanical and/or chemical techniques. For example, the surface can be roughened or patterned by rubbing, etching, grooving, stretching, and the oblique deposition of metal films. The substrate can be patterned using techniques such as photolithography (Kleinfield et al, J. Neurosci. 8: 4098-120 (1998)), photoetching, chemical etching and microcontact printing (Kumar et al., Langmuir 10: 1498-511 (1994)). Other techniques for forming patterns on a substrate will be readily apparent to those of skill in the art.

The size and complexity of the pattern on the substrate is limited only by the resolution of the technique utilized and the purpose for which the pattern is intended. For example, using microcontact printing, features as small as 200 nm have been layered onto a substrate. See, Xia, Y.; Whitesides, G., J. Am. Chem. Soc. 117:3274-75 (1995). Similarly, using photolithography, patterns with features as small as 1 μm have been produced. See, Hickman et al., J. Vac. Sci. Technol. 12:607-16 (1994). Patterns that are useful in the present invention include those which comprise features such as wells, enclosures, partitions, recesses, inlets, outlets, channels, troughs, diffraction gratings and the like.

In an exemplary embodiment, the patterning is used to produce a substrate having a plurality of adjacent addressable features, wherein each of the features is separately identifiable by a detection means. In another exemplary embodiment, an addressable feature does not fluidically communicate with other adjacent features. Thus, an analyte, or other substance, placed in a particular feature remains substantially confined to that feature. In another preferred embodiment, the patterning allows the creation of channels through the device whereby fluids can enter and/or exit the device.

In those embodiments in which the Polymer, the linker arm or a combination thereof are printed onto the substrate, the pattern can be printed directly onto the substrate or, alternatively, a “lift off” technique can be utilized. In the lift off technique, a patterned resist is laid onto the substrate, component of the chip is laid down in those areas not covered by the resist and the resist is subsequently removed: resists are known to those of skill in the art. See, for example, Kleinfield et al., J. Neurosci. 8:4098-120 (1998). In some embodiments, following removal of the resist, a second chip component, having a structure different from the first component layer is printed onto the substrate on those areas initially covered by the resist; a process that can be repeated any selected number of times with different components to produce a chip having a desired format.

Using the technique set forth above, substrates with patterns having regions of different chemical characteristics can be produced. Thus, for example, a pattern having an array of adjacent isolated features is created by varying the hydrophobicity/hydrophilicity, charge or other chemical characteristics of the pattern constituents. For example, hydrophilic compounds can be confined to individual hydrophilic features by patterning “walls” between the adjacent features using hydrophobic materials. Similarly, positively or negatively charged compounds can be confined to features having “walls” made of compounds with charges similar to those of the confined compounds. Similar substrate configurations are also accessible through microprinting a layer with the desired characteristics directly onto the substrate. See, Mrkish, M.; Whitesides, G. M., Ann. Rev. Biophys. Biomol. Struct. 25:55-78 (1996).

The specificity and multiplexing capacity of the chips of the invention can be increased by incorporating spatial encoding (e.g., spotted microarrays) into the chip substrate. Spatial encoding can be introduced into each of the chips of the invention. In an exemplary embodiment, binding functionalities for different analytes can be arrayed across the chip surface, allowing specific data codes (e.g., analyte-binding functionality specificity) to be reused in each location. In this case, the array location is an additional encoding parameter, allowing the detection of a virtually unlimited number of different analytes.

In the embodiments of the invention in which spatial encoding is utilized, they preferably utilize a spatially encoded array comprising m binding functionalities distributed over m regions of the substrate. Each of the m binding functionalities can be a different functionality or the same functionality, or different functionalities can be arranged in patterns on the surface. For example, in the case of Polymer array of addressable locations, all the locations in a single row or column can have the same binding functionality. The m binding functionalities are preferably patterned on the substrate in a manner that allows the identity of each of the m locations to be ascertained. In a preferred embodiment, the m binding functionalities are ordered in a p by q Polymer of (p×q) discrete locations, wherein each of the (p×q) locations has bound thereto at least one of the m binding functionalities. The microarray can be patterned from essentially any type of binding functionality.

The spatially encoded assay substrates can include essentially any number of compounds. In an embodiment in which the binding functionalities are polynucleotides (oligonucleotides or nucleic acids) or polypeptides, m is a number from 1 to 100, more preferably, from 10 to 1,000, and more preferably from 100 to 10,000.

In a particularly preferred embodiment, the substrate includes an aluminum support that is coated with a layer of silicon dioxide. In yet a further preferred embodiment, the silicon dioxide layer is from about 1000-3000 Å in thickness.

Those of skill in the art will appreciate that the above-described and other methods are useful for preparing arrays of a wide variety of compounds in addition to nucleic acids, are useful for preparing arrays of a wide variety of compounds in addition to nucleic acids.

Quality Control of Chips

For the first time, the present invention provides a simple, rapid method for performing quality control on biochips. The quality control is useful to ascertain the extent of coverage by the luminescent polymer of selected areas of the chip. The quality control process is compatible with high-throughput methodologies.

In an exemplary embodiment, a fluorescence spectrum of a chip is recorded using an appropriate method. Any appropriate method of detecting luminescence can be utilized in performing the quality control operations on the chips of the invention. The choice of a proper detection system for a particular application is well within the abilities of one of skill in the art. Exemplary detection means include, but are not limited to, detection by unaided eye, light microscopy using the eye or an optical sensor as the detector, confocal microscopy, laser scanning confocal microscopy, imaging using quantum dot color, fluorescence spectrum or other quantum dot property and wide-field imaging with a 2D CCD camera. In an exemplary embodiment, the device is a fluorescent plate reader.

In practicing the method, a fluorescence threshold for the plates or the individual regions on a plate is established. The threshold can be any useful number. The fluorescence is detected and optionally quantified. The data acquired from the detection can be utilized raw or submitted to further processing, e.g. smoothing, derivatizing, integrating, etc. Plates having fluorescence values above the threshold are designated “Pass,” those having fluorescence below the threshold are designated “Fail.”

Analytes

The device and methods of the present invention can be used to detect any analyte, or class of analytes, which interact with a binding functionality in a detectable manner. The interaction between the analyte and binding functionality can be any physicochemical interaction, including covalent bonding, ionic bonding, hydrogen bonding, van der Waals interactions, attractive electronic interactions and hydrophobic/hydrophilic interactions.

In an exemplary embodiment, the interaction is an ionic interaction. In this embodiment, an acid, base, metal ion or metal ion-binding ligand is the analyte. In a further exemplary embodiment, the interaction is a hydrogen bonding interaction.

In a preferred embodiment, the analyte molecule is a biomolecule such as a polypeptide (e.g., peptide or protein), a polynucleotide (e.g., oligonucleotide or nucleic acid), a carbohydrate (e.g., simple or complex carbohydrate) or a lipid (e.g., fatty acid or polyglycerides, phospholipids, etc.). In the case of proteins, the nature of the analyte can depend upon the nature of the binding functionality. For example, one can capture a ligand using a receptor for the ligand as a binding functionality; an antigen using an antibody against the antigen, or a substrate using an enzyme that acts on the substrate.

The analyte can be derived from any sort of biological source, including body fluids such as blood, serum, saliva, urine, seminal fluid, seminal plasma, lymph, and the like. It also includes extracts from biological samples, such as cell lysates, cell culture media, or the like. For example, cell lysate samples are optionally derived from, e.g., primary tissue or cells, cultured tissue or cells, normal tissue or cells, diseased tissue or cells, benign tissue or cells, cancerous tissue or cells, salivary glandular tissue or cells, intestinal tissue or cells, neural tissue or cells, renal tissue or cells, lymphatic tissue or cells, bladder tissue or cells, prostatic tissue or cells, urogenital tissues or cells, tumoral tissue or cells, tumoral neovasculature tissue or cells, or the like.

In another embodiment, the analyte is a member selected from the group consisting of acids, bases, organic ions, inorganic ions, pharmaceuticals, herbicides, pesticides, and noxious gases. Each of these analytes can be detected as a vapor or a liquid. The analyte can be present as a component in a mixture of structurally unrelated compounds, an assay mixture, racemic mixtures of stereoisomers, non-racemic mixtures of stereoisomers, mixtures of diastereomers, mixtures of positional isomers or as a pure compound. Within the scope of the invention is method to detect a particular analyte of interest without interference from other substances within a mixture.

The analyte can be labeled with a fluorophore or other detectable group either directly or indirectly through interacting with a second species to which a detectable group is bound. When a second labeled species is used as an indirect labeling agent, it is selected from any species that is known to interact with the analyte species. Preferred second labeled species include, but are not limited to, antibodies, aptazymes, aptamers, streptavidin, and biotin.

The analyte can be labeled either before or after it interacts with the binding functionality. The analyte molecule can be labeled with a detectable group or more than one detectable group. Where the analyte species is multiply labeled with more than one detectable group, the groups are preferably distinguishable from each other. Properties on the basis of which the individual quantum dots can be distinguished include, but are not limited to, fluorescence wavelength, absorption wavelength, fluorescence emission, fluorescence absorption, ultraviolet light absorbance, visible light absorbance, fluorescence quantum yield, fluorescence lifetime, light scattering and combinations thereof.

Organic ions, which are substantially non-acidic and non-basic (e.g., quaternary alkylammonium salts) can be detected by a binding functionality. For example, a binding functionality with ion exchange properties is useful in the present invention. A specific example is the exchange of a cation such as dodecyltrimethylammonium cation for a metal ion such as sodium, using a spacer arm presenting a negatively charged species. Binding functionalities that form inclusion complexes with organic cations are also of use. For example, crown ethers and cryptands can be used to form inclusion complexes with organic ions such as quaternary ammonium cations.

Inorganic ions such as metal ions and complex ions (e.g., SO₄⁻², PO₄⁻³) can also be detected using the device and method of the invention. Metal ions can be detected, for example, by their complexation or chelation by agents bound to the adsorbent layer. In this embodiment, the binding functionality can be a simple complexing moiety (e.g., carboxylate, amine, thiol) or can be a more structurally complicated agent (e.g., ethylenediaminepentaacetic acid, crown ethers, aza crowns, thia crowns).

Complex inorganic ions can be detected by, for example, their ability to compete with ligands for bound metal ions in ligand-metal complexes. When a ligand bound to a spacer arm or a substrate forms a metal-complex having a thermodynamic stability constant, which is less than that of the complex between the metal and the complex ion, the complex ion will replace the metal ion on the immobilized ligand. Methods of determining stability constants for compounds formed between metal ions and ligands are well known to those of skill in the art. Using these stability constants, substrates including affinity moieties that are specific for particular ions can be manufactured. See, Martell, A. E., Motekaitis, R. J., DETERMINATION AND USE OF STABILITY CONSTANTS, 2d Ed., VCH Publishers, New York 1992.

Small molecules such as pesticides, herbicides, and the like can be detected by the use of a number of different binding functionality motifs. Acidic or basic components can be detected as described above. An analyte's metal binding capability can also be used to advantage, as described above for complex ions. Additionally, if these analytes bind to an identified biological structure (e.g., a receptor), the receptor can be immobilized on the substrate, a spacer arm. Techniques are also available in the art for raising antibodies which are highly specific for a particular species. Thus, it is within the scope of the present invention to make use of antibodies against small molecules, pesticides, agents of war and the like for detection of those species. Techniques for raising antibodies to herbicides and pesticides are known to those of skill in the art. See, Harlow, Lane, MONOCLONAL ANTIBODIES: A LABORATORY MANUAL, Cold Springs Harbor Laboratory, Long Island, N.Y., 1988.

In another exemplary embodiment, the analyte is detected by binding to an immobilized binding functionality is an organophosphorous compound such as an insecticide.

Method of Making the Chip

In another aspect, the present invention provides methods of making a polymeric compound and a chip of the invention. As discussed above, the polymeric compound may be formed from substantially any appropriate luminescent moiety or combination of luminescent moieties. Cross-linked polymers are also useful as polymeric compounds of the invention.

The method of forming a chip of the invention includes depositing onto a substrate a polymer that includes an luminescent polymer having analyte-receiving properties as set forth above. The polymer can be formed in situ on the chip or prior to its deposition onto the chip.

Thus, in an exemplary embodiment, the invention provides a method of making a device for use in conjunction with a laser desorption analysis of an analyte molecule. The method includes contacting a surface of a substrate with a polymeric precursor including a luminescent polymerizable monomeric precursor of a luminescent polymer; and a polymerizable monomer that is not luminescent, or which is not usefully luminescent. The mixture of monomers is polymerized, thereby forming the layer of luminescent polymer. The polymeric compound is generally attached to the substrate via a chemical or physical interaction.

In an exemplary embodiment, the chip of the invention is washed after the polymeric matrix of the invention is deposited onto the substrate surface. The washing process is practiced with a solvent such as water or an organic solvent, e.g., alcohol, ether, ester, DMF, halocarbon (e.g., CH₂Cl₂, HCCl₃, CCl₄), amide, etc. The washing process is useful, for example, to remove reagents, reactants and small or incompletely polymerized species from the chip, or to cause the polymeric material to swell or contract.

In the method set forth above, the luminescent polymer optionally includes a third species incorporated into the polymer, having a structure different from the either of the other two species. For example, the third polymeric species can be selected from a second luminescent species, an analyte binding species, a cross-linking species and a combination thereof.

Assays

The chip of the present invention is useful in performing assays of substantially any format including, but not limited to chromatographic capture, immunoassays, competitive assays, DNA or RNA binding assays, fluorescence in situ hybridization (FISH), protein and nucleic acid profiling assays, sandwich assays and the like.

Thus, in a further aspect, the present invention provides a method of analyzing a sample. The method includes desorbing and ionizing the sample from a chip that includes a luminescent polymeric compound of the invention. The polymeric compound is a discrete polymer that is either formed prior to its deposition onto the chip or, alternatively, is formed in situ on the chip.

The following discussion focuses on the use of the methods of the invention in practicing exemplary assays. This focus is for clarity of illustration only and is not intended to define or limit the scope of the invention. Those of skill in the art will appreciate that the method of the invention is broadly applicable to any assay technique for detecting the presence and/or amount of a target.

The chip of the present invention is useful for performing retentate chromatography. Retentate chromatography has many uses in biology and medicine. These uses include combinatorial biochemical separation and purification of analytes, protein profiling of biological samples, the study of differential protein expression and molecular recognition events, diagnostics and drug discovery. Retentate chromatography is described in Hutchens and Yip, U.S. Pat. No. 6,225,047.

One basic use of retentate chromatography as an analytical tool involves exposing a sample to a combinatorial assortment of different adsorbent/eluant combinations and detecting the behavior of the analyte under the different conditions. This both purifies the analyte and identifies conditions useful for detecting the analyte in a sample. Substrates having adsorbents identified in this way can be used as specific detectors of the analyte or analytes. In a progressive extraction method, a sample is exposed to a first adsorbent/eluant combination and the wash, depleted of analytes that are adsorbed by the first adsorbent, is exposed to a second adsorbent to deplete it of other analytes. Selectivity conditions identified to retain analytes also can be used in preparative purification procedures in which an impure sample containing an analyte is exposed, sequentially, to adsorbents that retain it, impurities are removed, and the retained analyte is collected from the adsorbent for a subsequent round. See, for example, U.S. Pat. No. 6,225,047.

The chip of the invention is useful in applications such as sequential extraction of analytes from a solution, progressive resolution of analytes in a sample, preparative purification of an analyte, making probes for specific detection of analytes, methods for identifying proteins, methods for assembling multimeric molecules, methods for performing enzyme assays, methods for identifying analytes that are differentially expressed between biological sources, methods for identifying ligands for a receptor, methods for drug discovery (e.g., screening assays), and methods for generating agents that specifically bind an analyte.

In other applications, chip-based assays based on specific binding reactions are useful to detect a wide variety of targets such as drugs, hormones, enzymes, proteins, antibodies, and infectious agents in various biological fluids and tissue samples. In general, the assays consist of a target, a binding functionality for the target, and a means of detecting the target after its immobilization by the binding functionality (e.g., a detectable label). Immunological assays involve reactions between immunoglobulins (antibodies), which are capable of binding with specific antigenic determinants of various compounds and materials (antigens). Other types of reactions include binding between avidin and biotin, protein A and immunoglobulins, lectins and sugar moieties and the like. See, for example, U.S. Pat. No. 4,313,734, issued to Leuvering; U.S. Pat. No. 4,435,504, issued to Zuk; U.S. Pat. Nos. 4,452,901 and 4,960,691, issued to Gordon; and U.S. Pat. No. 3,893,808, issued to Campbell.

The present invention provides a chip useful for performing assays that are useful for confirming the presence or absence of a target in a sample and for quantitating a target in a sample. An exemplary assay format with which the invention can be used is an immunoassay, e.g., competitive assays, and sandwich assays. The invention is further illustrated using these two assay formats. The focus of the following discussion on competitive assays and sandwich assays is for clarity of illustration and is not intended to either define or limit the scope of the invention. Those of skill in the art will appreciate that the invention described herein can be practiced in conjunction with a number of other assay formats.

In an exemplary competitive binding assay, two species, one of which is the target, compete for a binding functionality on an adsorbent film. After an incubation period, unbound materials are washed off and the amount of target, or other species bound to the functionality is compared to reference amounts for determination of the target, or other species concentration in the assay mixture. Other competitive assay motifs using labeled target and/or labeled binding functionality and/or labeled reagents will be apparent to those of skill in the art.

A second type of assay is known as a sandwich assay and generally involves contacting an assay mixture with a surface having immobilized thereon a first binding functionality immunologically specific for that target. A second solution comprising a detectable binding material is then added to the assay. The labeled binding material will bind to a target, which is bound to the binding functionality. The assay system is then subjected to a wash step to remove labeled binding material, which failed to bind with the target and the amount of detectable material remaining on the chip is ordinarily proportional to the amount of bound target. In representative assays one or more of the target, binding functionality or binding material is labeled with a fluorescent label.

In addition to detecting an interaction between a binding functionality and a target, it is frequently desired to quantitate the magnitude of the affinity between two or more binding partners. The format of an assay for extracting affinity data for two molecules can be understood by reference to an embodiment in which a ligand that is known to bind to a receptor is displaced by an antagonist to that receptor. Other variations on this format will be apparent to those of skill in the art. The competitive format is well known to those of skill in the art. See, for example, U.S. Pat. Nos. 3,654,090 and 3,850,752.

The binding of an antagonist to a receptor can be assayed by a competitive binding method using a ligand for that receptor and the antagonist. One of the three binding partners (i.e., the ligand, antagonist or receptor) is bound to the binding functionality, or is the binding functionality. In an exemplary embodiment, the receptor is bound to the adsorbent film. Various concentrations of ligand are added to different chip regions. A detectable antagonist is then applied to each region to a chosen final concentration. The treated chip will generally be incubated at room temperature for a preselected time. The receptor-bound antagonist can be separated from the unbound antagonist by filtration, washing or a combination of these techniques. Bound antagonist remaining on the chip can be measured as discussed herein. A number of variations on this general experimental procedure will be apparent to those of skill in the art.

Competition binding data can be analyzed by a number of techniques, including nonlinear least-squares curve fitting procedure. When the ligand is an antagonist for the receptor, this method provides the IC50 of the antagonist (concentration of the antagonist which inhibits specific binding of the ligand by 50% at equilibrium). The IC50 is related to the equilibrium dissociation constant (Ki) of the antagonist based on the Cheng and Prusoff equation: Ki=IC50/(1+L/Kd), where L is the concentration of the ligand used in the competitive binding assay, and Kd is the dissociation constant of the ligand as determined by Scatchard analysis. These assays are described, among other places, in Maddox et al., J Exp Med., 158: 1211 (1983); Hampton et al., SEROLOGICAL METHODS, A LABORATORY MANUAL, APS Press, St. Paul, Minn., 1990.

The chip and method of the present invention are also of use in screening libraries of compounds, such as combinatorial libraries. The synthesis and screening of chemical libraries to identify compounds, which have novel bioactivities, and material science properties is now a common practice. Libraries that have been synthesized include, for example, collections of oligonucleotides, oligopeptides, and small and large molecular weight organic or inorganic molecules. See, Moran et al., PCT Publication WO 97/35198, published Sep. 25, 1997; Baindur et al., PCT Publication WO 96/40732, published Dec. 19, 1996; Gallop et al, J. Med. Chem. 37:1233-51 (1994).

Virtually any type of compound library can be probed using the method of the invention, including peptides, nucleic acids, saccharides, small and large molecular weight organic and inorganic compounds. In a presently preferred embodiment, the libraries synthesized comprise more than 10 unique compounds, preferably more than 100 unique compounds and more preferably more than 1000 unique compounds.

The nature of these libraries is better understood by reference to peptide-based combinatorial libraries as an example. The present invention is useful for assembling peptide-based combinatorial libraries, but it is not limited to these libraries. The methods of the invention can be used to screen libraries of essentially any molecular format, including small organic molecules, carbohydrates, nucleic acids, polymers, organometallic compounds and the like. Thus, the following discussion, while focusing on peptide libraries, is intended to be illustrative and not limiting.

Libraries of peptides and certain types of peptide mimetics, called “peptoids”, are assembled and screened for a desirable biological activity by a range of methodologies (see, Gordon et al., J. Med. Chem., 37: 1385-1401 (1994); Geysen, (Bioorg. Med. Chem. Letters, 3: 397-404 (1993); Proc. Natl. Acad. Sci. USA, 81: 3998 (1984); Houghton, Proc. Natl. Acad. Sci. USA, 82: 5131 (1985); Eichler et al., Biochemistry, 32: 11035-11041 (1993); and U.S. Pat. No. 4,631,211); Fodor et al., Science, 251: 767 (1991); Huebner et al. (U.S. Pat. No. 5,182,366). Small organic molecules have also been prepared by combinatorial means. See, for example, Camps. et al., Annaks de Quimica, 70: 848 (1990); U.S. Pat. No. 5,288,514; U.S. Pat. No. 5,324,483; Chen et al., J. Am. Chem. Soc., 116: 2661-2662 (1994).

In an exemplary embodiment, a binding domain of a receptor, for example, serves as the focal point for a drug discovery assay, where, for example, the receptor is immobilized, and incubated both with agents (i.e., ligands) known to interact with the binding domain thereof, and a quantity of a particular drug or inhibitory agent under test. The extent to which the drug binds with the receptor and thereby inhibits receptor-ligand complex formation can then be measured. Such possibilities for drug discovery assays are contemplated herein and are considered within the scope of the present invention. Other focal points and appropriate assay formats will be apparent to those of skill in the art.

In each of the assays set forth above, a washing step or steps is optionally incorporated. The washing step(s) can be performed before the chip is contacted with the analyte and/or after the chip is contacted with the analyte. In a still further exemplary embodiment, the chip of the invention is washed after the polymeric matrix of the invention is deposited onto the substrate surface. The washing process is practiced with a solvent such as water or an organic solvent, e.g., alcohol, ether, ester, DMF, halocarbon (e.g., CH₂Cl₂, HCCl₃, CCl₄), amide, etc. The choice of solvent is dependent on the polymer and the polymer and the analyte if the washing is performed subsequent to contacting the polymer with the analyte. The choice of the correct solvent for a particular application is well within the abilities of those of skill in the art. The washing process is useful, for example, to remove reagents, reactants and small or incompletely polymerized species from the chip, or to cause the polymeric matrix to swell or contract. Moreover, the washing process can be used to remove components of the assay mixture that interfere with the analysis, and which are amenable to removal from the chip under conditions that allow the desired analyte mixture component(s) to continue to interact with the chip.

Detection

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

In another embodiment, a fluorescent modifying agent is used to label one or more assay component or region of the chip. In this embodiment, when the fluorescence modifying agent is in operative proximity to the lumophore of the polymer, the modifying agent alters a fluorescence property of the lumophore. The modification generally occurs through energy transfer between the lumophore and the modifying agent, such as fluorescence resonance energy transfer. A representative example of an energy transfer pair is a lumophore and a quencher. In another embodiment, the polymer is labeled with a fluorescence modifying agent and the analyte is labeled with a lumophore.

The alteration in the fluorescence property allows the binding of the analyte to the polymer to be detected. For example, if a decrease in luminescence of the polymer is detected following contacting the polymer with an analyte that includes a quencher, the decrease is indicative that the analyte is interacting with the polymer.

Microscopic techniques of use in practicing the invention include, but are not limited to, simple light microscopy, confocal microscopy, polarized light microscopy, atomic force microscopy (Hu et al., Langmuir 13:5114-5119 (1997)), scanning tunneling microscopy (Evoy et al., J. Vac. Sci. Technol A 15:1438-1441, Part 2 (1997)), and the like.

Spectroscopic techniques of use in practicing the present invention include, for example, infrared spectroscopy (Zhao et al., Langmuir 13:2359-2362 (1997)), raman spectroscopy (Zhu et al., Chem. Phys. Lett. 265:334-340 (1997)), X-ray photoelectron spectroscopy (Jiang et al., Bioelectroch. Bioener. 42:15-23 (1997)) and the like. Visible and ultraviolet spectroscopies are also of use in the present invention.

Other useful techniques include, for example, surface plasmon resonance (Evans et al., J. Phys. Chem. B 101:2143-2148 (1997), ellipsometry (Harke et al., Thin Solid Films 285:412-416 (1996)), impedometric methods (Rickert et al., Biosens. Bioelectron. 11:757:768 (1996)), and the like.

In addition, the Polymerase Chain Reaction (PCR) and other related techniques have gained wide use for amplifying the number of nucleic acid analytes in a sample. By the addition of appropriate enzymes, reagents, and temperature cycling methods, the number of nucleic acid analyte molecules are amplified such that the analyte can be detected by most known detection means.

Of particular interest is the use of mass spectrometric techniques to detect analytes interacting with the luminescent, particularly those mass spectrometric methods utilizing desorption of the analyte from the polymer and direct detection of the desorbed analytes.

Desorbing the analyte from the luminescent polymer involves exposing the analyte to an appropriate energy source. Usually this means striking the analyte with radiant energy or energetic particles. For example, the energy can be light energy in the form of laser energy (e.g., UV laser) or energy from a flash lamp. Alternatively, the energy can be a stream of fast atoms. Heat may also be used to induce/aid desorption.

The luminescent polymer and biochips of this invention can also be useful for Both MALDI and SELDI. MALDI for large proteins is described in, e.g., U.S. Pat. No. 5,118,937 (Hillenkamp et al.) and U.S. Pat. No. 5,045,694 (Beavis and Chait). SELDI is described in U.S. Pat. No. 5,719,060 (Hutchens and Yip). SELDI is a method for desorption in which the analyte is presented to the energy stream on a surface that captures the analyte and, thereby, enhances analyte capture and/or desorption.

One version of SELDI, called SEAC (Surface-Enhanced Affinity Capture), involves presenting the analyte to the desorbing energy in association with an affinity capture device (i.e., an adsorbent) attached to probe surface. When an analyte is so adsorbed, the desorbing energy source is provided with a greater opportunity to desorb the target analyte. An energy absorbing material, e.g., matrix, usually is added to the probe to aid desorption of biomolecules, prior to presenting the probe to the energy source, e.g., laser, for desorbing the analyte. Typically used matrix materials include sinapinic acid (SPA) and alpha-cyano-4-hydroxy cinnamic acid (CHCA).

The desorbed analyte can be detected by any of several means. When the analyte is ionized in the process of desorption, such as in laser desorption/ionization mass spectrometry, the detector can be an ion detector. Mass spectrometers generally include means for determining the time-of-flight of desorbed ions. This information is converted to mass. One need not determine the mass of desorbed ions, however, to resolve and detect them: the fact that ionized analytes strike the detector at different times provides detection and resolution of them.

A plurality of detection means can be implemented in series to fully interrogate the analyte components and function associated with retentate at each location in the array.

Desorption detectors comprise means for desorbing the analyte from the adsorbent and means for directly detecting the desorbed analyte. That is, the desorption detector detects desorbed analyte without an intermediate step of capturing the analyte in another solid phase and subjecting it to subsequent analysis. Detection of an analyte normally will involve detection of signal strength. This, in turn, reflects the quantity of analyte adsorbed to the adsorbent.

The desorption detector also can include other elements, e.g., a means to accelerate the desorbed analyte toward the detector, and a means for determining the time-of-flight of the analyte from desorption to detection by the detector.

A preferred desorption detector is a laser desorption/ionization mass spectrometer, which is well known in the art. The mass spectrometer includes a port into which the substrate that carries the adsorbed analytes, e.g., a probe, is inserted. Striking the analyte with energy, such as laser energy desorbs the analyte. Striking the analyte with the laser results in desorption of the intact analyte into the flight tube and its ionization. The flight tube generally defines a vacuum space. Electrified plates in a portion of the vacuum tube create an electrical potential which accelerate the ionized analyte toward the detector. A clock measures the time of flight and the system electronics determines velocity of the analyte and converts this to mass. As any person skilled in the art understands, any of these elements can be combined with other elements described herein in the assembly of desorption detectors that employ various means of desorption, acceleration, detection, measurement of time, etc. An exemplary detector further includes a means for translating the surface so that any spot on the array is brought into line with the laser beam.

Data Processing

Data generation in mass spectrometry begins with the detection of ions by an ion detector. A typical laser desorption mass spectrometer can employ a nitrogen laser at 337.1 nm. A useful pulse width is about 4 nanoseconds. Generally, power output of about 1-25 μJ is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

A computer can transform the resulting spectrum into various formats for displaying. In one format, referred to as “spectrum view or retentate map,” a standard spectral view can be displayed, wherein the view depicts the quantity of analyte reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes that are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

The spectra that are generated in embodiments of the invention can be classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002 0193950 A1 (Gavin et al., “Method or analyzing mass spectra,” Dec. 19, 2002); U.S. 2003 0004402 A1 (Hitt et al., “Process for discriminating between biological states based on hidden patterns from biological data,” Jan. 2, 2003); and U.S. 2003 0055615 A1 (Zhang and Zhang, “Systems and methods for processing biological expression data” Mar. 20, 2003).

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

Informatics

As high-resolution, high-sensitivity datasets acquired using the methods of the invention become available to the art, significant progress in the areas of diagnostics, therapeutics, drug development, biosensor development, and other related areas will occur. For example, disease markers can be identified and utilized for better confirmation of a disease condition or stage (see, U.S. Pat. Nos. 5,672,480; 5,599,677; 5,939,533; and 5,710,007). Subcellular toxicological information can be generated to better direct drug structure and activity correlation (see, Anderson, L., “Pharmaceutical Proteomics: Targets, Mechanism, and Function,” paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see, U.S. Pat. No. 5,811,231). Similar advantages accrue from datasets relevant to other biomolecules and bioactive agents (e.g., nucleic acids, saccharides, lipids, drugs, and the like).

Thus, in another preferred embodiment, the present invention provides a database that includes at least one set of data assay data. The data contained in the database is acquired using a method of the invention and/or a QD-labeled species of the invention either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases, which include peptide sequence specificity data is for clarity of illustration only. It will be apparent to those of skill in the art that similar databases can be assembled for any assay data acquired using an assay of the invention.

The compositions and methods described herein for identifying and/or quantitating the relative and/or absolute abundance of a variety of molecular and macromolecular species from a biological sample provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

The present invention provides a computer database comprising a computer and software for storing in computer-retrievable form assay data records cross-tabulated, for example, with data specifying the source of the target-containing sample from which each sequence specificity record was obtained.

In an exemplary embodiment, at least one of the sources of target-containing sample is from a tissue sample known to be free of pathological disorders. In a variation, at least one of the sources is a known pathological tissue specimen, for example, a neoplastic lesion or a tissue specimen containing a pathogen such as a virus, bacteria or the like. In another variation, the assay records cross-tabulate one or more of the following parameters for each target species in a sample: (1) a unique identification code, which can include, for example, a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected binding functionality) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

The materials, methods and devices of the present invention are further illustrated by the examples, which follow. These examples are offered to illustrate, but not to limit the claimed invention.

### EXAMPLES

Materials and Methods

In the examples below, unless otherwise stated, temperatures are given in degrees Celsius (° C.); operations were carried out at room or ambient temperature (typically a range of from about 18-25° C.; evaporation of solvent was carried out using a rotary evaporator under reduced pressure (typically, 4.5-30 mmHg) with a bath temperature of up to 60° C.; the course of reactions was typically followed by TLC and reaction times are provided for illustration only; melting points are uncorrected; products exhibited satisfactory ¹H-NMR and/or microanalytical data; yields are provided for illustration only; and the following conventional abbreviations are also used: mp (melting point), L (liter(s)), mL (milliliters), mmol (millimoles), g (grams), mg (milligrams), min (minutes), and h (hours).

The efficacy of the polymeric EAM of the invention was assessed using a buffered peptide mixture containing approximately 4 μM vasopressin, 2 μM somatostatin, 4 μM insulin B-chain, 7 μM h,r-insulin and 5 μM hirudin. Approximately 1-2 μL of the peptide mixture was deposited onto the chip at each spot having the polymer film as described below.

### Example 1

Synthesis of an Anthracenyl-Polymer

A 5% initiator solution was prepared by dissolving of 2-hydroxy-4-(2-hydroxyethoxy)-2-methylpropiophenone (0.2500 g±0.005 g) in dimethylsulfoxide (5.0 g) in a 10 mL amber vial. The mixture was sonicated for 5-10 seconds to mix well

A functional monomer stock solution was prepared by combining glycerol (4.0 g+0.04 g), [3-(methacryloylamino)propyl]trimethylammonium chloride (6.0 g±0.03 g), 2% N, N-methylenbisacrylamide (6.0 g±0.03 g), de-ionized water (8.0 g±0.05 g) in a 20 mL amber vial. The mixture was sonicated to mix well.

A 0.2% fluorescence monomer (FM) stock solution was prepared by combining in a 5 mL amber vial add following: 9-anthracenylmethyl acrylate (0.003 g±0.01 g); and dimethylsulfoxide (1500 μL). The mixture was sonicated to mix well.

A working monomer solution was prepared by mixing the functional monomer stock solution (1.0 g±0.01 g), ethanol (6.6 g±0.05 g), the 5% initiator solution (100 μL), and the 0.2% FM stock solution (100 μL). The vial was shaken to mix well and sonicated for one minute.

### Example 2

Chip Preparation

Grit blast MA-CVD chips were arranged in universal racks. Prior to depositing monomer on the chip, the Cartesian was set up to deliver for two or more universal racks at a time and to deposit 1.5 μL of working monomer solution per spot. The monomer was deposited on the chip. After depositing the monomer, the arrays were allowed to sit in the Cartesian humid chamber for 2 minutes. The arrays were then moved to the large UV chamber purging was started. The chamber was purged at flow meter setting 30±2 for two minutes.

The UV cure was stared after two minutes and the arrays were cured for 10 minutes at UV intensities ˜5-6 while the gas purge is continued. Following the cure, the chips are cleaned by washing the chips for 5 minutes with 1M NaCl at 70 rpm. The chips were then washed two times with deionized water for 2 minutes each time at 70 rpm. After the deionized water wash, the chip racks were placed in and oven pre-set to 60° C. for 20 minutes to dry. When the chips were dry, they were loaded into the array cassettes.

### Example 3

Chip Quality Control Assay

The array cassette containing the chips was loaded into the Fluorometer (fluorescence microplate reader) for the reading, 12 arrays at a time. The arrays were read using endpoint on spot reading with excitation wavelength 260 and emission wavelength 420 with auto cutoff point. The Fluorometer data was transferred to an Excel template and it was determined whether the arrays passed or failed the test. Any failed arrays were removed and the arrays were repacked to provide complete set of 12 arrays per cassette.

The anthracene-containing polymer was detected with the excitation wavelength set at 260 nm, with an emission maximum at 420 nm.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A device comprising:
(a) a substrate having a surface; and
(b) a copolymer attached to said surface wherein said copolymer is formed from a luminescent monomer and a non-luminescent monomer.

2. The device according to claim 1, wherein the luminescent monomer is a fluorescent monomer which, when incorporated into the polymer, has a quantum yield of greater than 0.2 at 25° C.

3. The device according to claim 1, wherein said non-luminescent monomer, when incorporated into the polymer, has a quantum yield of less than 0.2 at 25° C.

4. The device according to claim 1, wherein said luminescent monomer and said non-luminescent monomer are acryloyl monomers.

5. The device according to claim 1, wherein said polymer comprises a subunit having the formula: 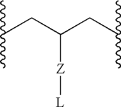
in which
Z is a linker moiety that is a member selected from substituted or unsubstituted alkyl, substituted or unsubstituted heteroalkyl, and substituted or unsubstituted aryl; and
L is a luminescent moiety.

6. The device according to claim 1, wherein said luminescent monomer has the formula:

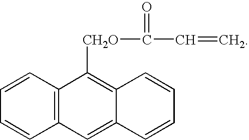

7. The device according to claim 6, wherein said polymer comprises a subunit having the formula:

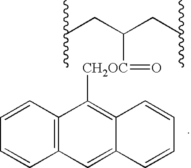

8. The device according to claim 1, wherein the polymeric material is attached to the surface by physical adhesion.

9. The device according to claim 1, wherein the polymer is attached to the surface covalently.

10. The device according to claim 1, wherein said polymer is a cross-linked polymer.

11. The device according to claim 1, wherein said polymeric material further comprises a functionality selected from an electrostatic functionality, a hydrophobic functionality, a hydrogen bonding functionality, a coordinate covalent bonding functionality, a covalent bonding functionality, a biospecific bonding functionality and combinations thereof.

12. The device according to claim 1, further comprising said analyte molecules adsorbed onto said polymer.

13. The device according to claim 1, wherein the substrate is in the form of a probe that is removably insertable into a mass spectrometer.

14. The device according to claim 13, wherein the polymer is attached to the substrate is in a plurality of addressable locations.

15. A method of detecting a molecular analyte comprising the steps of:
(a) contacting an analyte molecule with the polymer on the device of claim 1;
(b) striking the polymer with a high fluence energy pulse whereby the analyte is desorbed from the surface and ionized; and
(c) detecting the desorbed and ionized analyte molecule by mass spectrometry.

16. The method of claim 15 wherein the high fluence energy is laser energy.

17. A method of making a device for use in conjunction with a laser desorption analysis of an analyte molecule, said method comprising:
(a) contacting a surface of a substrate with a luminescent polymerizable monomer;
(b) contacting said surface with a non-luminescent polymerizable monomer;
(c) co-polymerizing said luminescent monomer and said non-luminescent monomer, thereby forming said layer of luminescent polymer;
(d) immobilizing said layer of said luminescent polymer on said surface.

18. A quality control method for a device for use in conjunction with a laser desorption analysis of an analyte molecule, said device comprising:
a substrate having a surface; and
a copolymer attached to said surface wherein said copolymer is formed from a luminescent monomer and a non-luminescent monomer;
said method comprising:
(a) selecting a threshold fluorescence intensity value for fluorescence emitted by said copolymer on said surface;
(b) determining said fluorescence intensity; and
(c) comparing said fluorescence intensity with said threshold fluorescence intensity value.
